SEQUENCE LISTING (4 sequences)

SEQ ID NO: 1 - protein, 437 aa, Homo sapiens
MSAVLLLALLGFILPLPGVQALLCQFGTVQHVWKVSDLPRQWTPKNTSCDSGLGCQDTLMLIESGPQVSLVLSKGCTEAKDQEPRVTEHRMGPGLSLISYTFVCRQEDFCNNLVNSLPLWAPQPPADPGSLRCPVCLSMEGCLEGTTEEICPKGTTHCYDGLLRLRGGGIFSNLRVQGCMPQPGCNLLNGTQEIGPVGMTENCNRKDFLTCHRGTTIMTHGNLAQEPTDWTTSNTEMCEVGQVCQETLLLLDVGLTSTLVGTKGCSTVGAQNSQKTTIHSAPPGVLVASYTHFCSSDLCNSASSSSVLLNSLPPQAAPVPGDRQCPTCVQPLGTCSSGSPRMTCPRGATHCYDGYIHLSGGGLSTKMSIQGCVAQPSSFLLNHTRQIGIFSAREKRDVQPPASQHEGGGAEGLESLTWGVGLALAPALWWGVVCPSC

SEQ ID NO: 2 - protein, 817 aa, Mus musculus
MNSIPVLTLLGVTALLPCVPALTCQKSSAQAVRNVAELPLRWWGAGEKTCEVSEGCQDLIMLLYNGPKVNLVIIKGCTEVEDQEPKVIWLRTGPGLSVVSYTRVCRHGDLCNDVNSTKILEELPTPTVPGSLRCPLCLSNDSCENAPEQVCPVGSTHCYDGVLRLRGDGIRTNLKVQGCMAQPDCNLLNGTQAIGTLYMSENCDLIGPQALDCNSGSLETVRNVSDLHLSWTTGWQTCEAGEGCYETVMLIQNGHEFHMVLTKGCTRDMNKKARLTRHRTGPGISIVSYVHVCRDRDFCNDLSTTDPLWTPPPDTELGTLRCRHCLSTGSCVSASELVCPAGSTHCYSGVLSLRGGGVISDLKVQGCISQSQPGCNLLNGTQTIGPVDVREDCGLQLDALKCQHGTLKTIQDISKLPLQWTAGQKICNVGEGCQDTLMLIENGEQVNLVLTKGCTTAKDQEAKVTEHRTGPGLSVTSYTRVCRKKDFCNDLSTTAPLWAPPPVTAPGTTRCPLCFSEQACENAPEQVCPAGSTHCYSGVLSLRGGGIISDLKVQGCMSQPGCNLLNGTQTIGPVDVSERCSPPSETTELSCYRGVMFELGNGFAEEPVKWTAPGSQVCAPDEICQETLLLIDVGQKSAFLGSKGCSSPGAQDNIGVSIFSRLPGMLVASYTKFCSSHLCNGADSSSVLLSILPRPDVPPPGDVQCPMCVELFGSCKSTDSVTCPRGATHCYKGDIALQGGGLTTRVSIQGCMAPPIKPLLGDSKTIGIFSAEESSNYRHEDDVTSAPSLAWTLRLSAWMLGLSALLSSLYAGICPLC

SEQ ID NO: 3 - DNA, 1314 bp, Homo sapiens
atgagcgcggtattactgctggccctcctggggttcatcctcccactgccaggagtgcaggcgctgctctgccagtttgggacagttcagcatgtgtggaaggtgtccgacctgccccggcaatggacccctaagaacaccagctgcgacagcggcttggggtgccaggacacgttgatgctcattgagagcggaccccaagtgagcctggtgctctccaagggctgcacggaggccaaggaccaggagccccgcgtcactgagcaccggatgggccccggcctctccctgatctcctacaccttcgtgtgccgccaggaggacttctgcaacaacctcgttaactccctcccgctttgggccccacagcccccagcagacccaggatccttgaggtgcccagtctgcttgtctatggaaggctgtctggaggggacaacagaagagatctgccccaaggggaccacacactgttatgatggcctcctcaggctcaggggaggaggcatcttctccaatctgagagtccagggatgcatgccccagccaggttgcaacctgctcaatgggacacaggaaattgggcccgtgggtatgactgagaactgcaataggaaagattttctgacctgtcatcgggggaccaccattatgacacacggaaacttggctcaagaacccactgattggaccacatcgaataccgagatgtgcgaggtggggcaggtgtgtcaggagacgctgctgctcatagatgtaggactcacatcaaccctggtggggacaaaaggctgcagcactgttggggctcaaaattcccagaagaccaccatccactcagcccctcctggggtgcttgtggcctcctatacccacttctgctcctcggacctgtgcaatagtgccagcagcagcagcgttctgctgaactccctccctcctcaagctgcccctgtcccaggagaccggcagtgtcctacctgtgtgcagccccttggaacctgttcaagtggctccccccgaatgacctgccccaggggcgccactcattgttatgatgggtacattcatctctcaggaggtgggctgtccaccaaaatgagcattcagggctgcgtggcccaaccttccagcttcttgttgaaccacaccagacaaatcgggatcttctctgcgcgtgagaagcgtgatgtgcagcctcctgcctctcagcatgagggaggtggggctgagggcctggagtctctcacttggggggtggggctggcactggccccagcgctgtggtggggagtggtttgcccttcctgctaa

SEQ ID NO: 4 - DNA, 2454 bp, Mus musculus
atgaattctataccagtgctgacccttctgggggtcacggccttgctaccctgtgtcccagctctgacctgccagaaaagcagcgcacaggctgtgaggaatgtggcagagctgcccctcaggtggtggggagctggtgagaaaacctgcgaggttagcgagggttgccaagacttgataatgctcctgtataatggacccaaggtcaacttggtgatcatcaagggctgcaccgaggttgaggaccaagagccgaaggtgatctggctcaggacaggccctgggctctctgtggtgtcctacacccgtgtgtgtcgccatggtgacctctgcaatgatgtgaacagcactaagatccttgaggagctacctacacccacagttccagggtccctgcgctgcccactctgcctttctaatgacagctgtgagaatgcaccggagcaggtctgccctgtgggaagcacacactgctacgatggagtcctcaggctcaggggagatggcatcaggaccaatctcaaggtgcagggctgcatggcccagccagactgcaacctgcttaatggcacccaggcgattgggaccttgtatatgagcgaaaactgtgatcttataggtccacaggctctggattgcaatagtgggagcttggaaactgtgaggaatgtatcagatctgcacttgagctggacgactggctggcaaacctgtgaggctggcgaggggtgttatgaaacagtgatgctaatacaaaatggacatgaatttcacatggttctcactaagggatgtactagggatatgaacaaaaaggctcggctcaccaggcatagaacaggcccagggatctccatcgtctcctacgtgcatgtgtgccgcgacagggacttctgtaatgacctgtctacaacagaccctctttggaccccgccccctgacacagagctagggaccctgcgctgccgacactgcctttcaaccggcagctgtgtgagtgcatccgagctggtctgccccgcaggcagcacacactgctacagtggagtcctcagcctcaggggaggaggggtcatttctgatctgaaggtacagggatgcatatcgcagtcccagccaggatgcaacctgctcaacggtacccagacaatcggacccgtggatgtgcgtgaggactgcggtcttcagttagatgctctgaaatgccagcatgggacgctgaagaccatccaggatatatcgaagctgcctctccagtggacggctggccagaaaatctgtaatgtgggtgaaggctgccaagacacactgatgttgatagagaacggagagcaggtgaacttggttctcacgaaaggctgcactaccgcaaaggaccaagaggccaaagtcacggagcacagaactggaccagggctgtctgtcacctcctacacccgagtgtgccgtaaaaaagacttctgcaatgacctgtctaccacagcccctctctgggctccacctccagtgacagccccagggaccactcgctgccctctctgcttttctgaacaagcctgtgagaatgcaccggagcaggtctgccctgcaggcagcacacactgctacagtggagtcctcagcctcaggggaggagggatcatctctgatctgaaggtgcagggctgtatgtcccagccaggatgcaacctgctcaacggtacccagacaatcggacccgtggatgtgagcgagcgctgcagtcctccgtcagaaacaacagagttgtcctgttacaggggtgtgatgtttgagcttggcaatggctttgcggaggaacctgtcaagtggacggcaccagggtctcaggtgtgtgcacctgatgagatttgtcaagagacgctgctgctcatagacgtaggacaaaaatcagccttcctggggagtaaaggctgcagcagtcctggggcgcaggacaatattggtgtctccatattctcccggctccctgggatgctggtagcttcctataccaaattctgttcttcccacctgtgcaatggagccgacagcagcagtgtccttctaagcatcctccctcgtccagatgttcctcccccaggagatgtgcagtgccccatgtgtgtagagttatttggatcctgcaagagcactgactctgtcacctgccctaggggtgccactcactgttataaaggtgacattgcactacagggaggtggactgactaccagagtgagcattcaggggtgcatggccccacctatcaaacctttactgggtgactccaaaacaatcggtatcttctcggcagaggagagctctaactatcgacatgaggatgatgttacctcggccccttccctggcctggaccttacggctatcggcctggatgttagggctatcggctcttctcagctctttgtatgctgggatctgtcctctctgctga

(19) United States
(12) Patent Application Publication
(10) Pub. No.: US 2010/0306863 A1
(43) Pub. Date: Dec. 2, 2010

(54) SCREENING, THERAPY AND DIAGNOSIS

(75) Inventors: Marco Colonna, St. Louis, MO (US); Julia Klesney-Tait, Iowa City, IA (US); Paola Panina, Milan (IT)

Correspondence Address: FOLEY AND LARDNER LLP;SUITE 500, 3000 K STREET NW, WASHINGTON, DC, 20007, US

(73) Assignee: Bioxell S.p.A.

(21) Appl. No.: 12/452,813

(22) PCT Filed: Jul. 23, 2008
(86) PCT No.: PCT/EP2008/059668

§ 371 Date: Aug. 5, 2010

Related U.S. Application Data
(60) Provisional application No. 60/961,615, filed on Jul. 23, 2007.

(51) Int. Cl. C12P 21/00; A01K 67/00; C07K 16/18; A61K 39/395; C12N 5/20; A61P 37/00

(52) U.S. Cl. 800/6; 800/13; 530/389.2; 424/158.1; 435/326

(57) ABSTRACT

A TREM-1 ligand is identified. This allows various derivatives to be provided/identified that are capable of binding to the TREM-1 receptor. The TREM-1 ligand or the derivatives can be used in screening for drugs/drug candidates. Substances that block or reduce binding of the TREM-1 ligand/derivative to a TREM-1 receptor may be useful for treating sepsis, particularly sepsis of bacterial or fungal origin. Antibodies to the ligand may be useful in diagnosing sepsis, particularly sepsis of bacterial or fungal origin.

The present invention relates to the diagnosis and treatment of inflammatory disorders. It also relates to screening methods for identifying drugs or drug candidates of potential use in treating inflammatory disorders and particularly sepsis and inflammatory bowel disease (IBD).

TREM-1 is a cell-surface molecule that has been identified both on human and murine polymorphonuclear neutrophils and mature monocytes [Bouchon et al, J. Immunol. 164: 4991-4995 (2000)]. It belongs to the immunoglobulin superfamily and activates downstream signalling pathways with the help of an adapter protein called DAP12 [Bouchon et al, supra; Colonna et al, J. Infect. Dis. 187 (Supply S297-301 (2003), Colonna Nat. Rev. Immunol. 6:445-453 (2003); Nathan et al Nat. Med. 7:530-2 (2001)].

Triggering via TREM-1 results in the production of pro-inflammatory cytokines, chemokines, reactive oxygen species, and leads to rapid degranulation of neutrophilic granules, and phagocytosis. It has been shown that blockade of TREM-1 signaling suppresses the development of collagen-induced arthritis (Abstract presented by Y. Murakami at: Innate Immunity: Signaling Mechanisms February 2008, Keystone, Colo.). Furthermore, TREM-1 activation, by dampening LPS-induced IL-12 family cytokines, may impact T cell responses in vivo, thus suggesting an in vivo role of TREM-1 activation not only in innate but also in adaptive immune responses (Dower K, J. Immunol. 2008 180:3520-3534). Since interfering with TREM-1 engagement leads to the simultaneous reduction in production and secretion of a variety of proinflammatory mediators, TREM-1 represents an attractive target for treating chronic inflammatory disorders. Indeed, a role for TREM-1 has been demonstrate in a variety of inflammatory disorders, including acute endotoxemia, Helicobacter pylori infection, hepatic granulomatosis, Salmonella enterica infection, Infectious lung diseases, Marburg and Ebola viruses infections, Acute respiratory distress syndrome (ARDS), inflammatory bowel disease and rheumatoid arthritis, sepsis.

Sepsis constitutes a significant consumption of intensive care resources and remains an ever-present problem in the intensive care unit. It has been estimated that between 400,000 and 500,000 patients are so affected each year in both the USA and Europe. Morbidity and mortality have remained high despite improvements in both supportive and anti-microbial therapies. Mortality rates vary from 40% for uncomplicated sepsis to 80% in those suffering from septic shock and multi-organ dysfunction. The pathogenesis of the conditions is now becoming better understood. Greater understanding of the complex network of immune, inflammatory and haematological mediators may allow the development of rational and novel therapies.

Following an infection, innate and cognitive immune responses develop in sequential phases that build-up in specificity and complexity, resulting ultimately in the clearance of infectious agents and restoration of homeostasis. The innate immune response serves as the first line of defence and is initiated upon activation of pattern recognition receptors, such as Toll-like receptors (TLRs) [Aderem et al, Nature 406:782-786 (2000) and Thoma-Uszynski et al, Science 291:1544-1549(2001)]. Activation occurs by various pathogen-associated microbial patterns (PAMPs) [Medzhitov et al, N. Engl. J. Med. 343:338-344 (2000)]. Activation of the TLRs triggers the release of large quantities of such cytokines as TNF-α and IL-1β, which, in case of such massive infections as sepsis, can precipitate tissue injury and lethal shock [Cohen, et al, Nature 420:885-91 (2002); Hotchkiss et al, N. Engl. J. Med. 348:138-50 (2003)].

Another receptor involved, inter alia, in response to infection is known as the “triggering receptor expressed on myeloid cells-1” (TREM-1). This is a member of a recently discovered family of receptors, the TREM family, expressed on the surface of neutrophils and a subset of monocytes. TREM receptors activate myeloid cells via association with the adaptor molecule DAP12. Engagement of TREM-1 has been reported to trigger the synthesis of pro-inflammatory cytokines in the presence of microbial products.

TREM-1 is a cell-surface molecule that has been identified both on human and murine polymorphonuclear neutrophils and mature monocytes [Bouchon et al, J. Immunol. 164: 4991-4995 (2000)]. It belongs to the immunoglobulin superfamily and activates downstream signalling pathways with the help of an adapter protein called DAP12 [Bouchon et al, supra; Colonna et al, J. Infect. Dis. 187 (Suppl): S297-301 (2003), Colonna Nat. Rev. Immunol. 6:445-453 (2003); Nathan et al Nat. Med. 7:530-2 (2001)].

Bouchon and co-workers have shown that the expression of TREM-1 was greatly up-regulated on neutrophils and monocytes in the presence of bacteria such as Pseudomonas aeruginosa or Staphylococcus aureus, both in cell culture and in tissue samples from patients with infection [Bouchon et al, Nature 410:1103-1107 (2001)]. In striking contrast, TREM-1 was not up-regulated in samples from patients with non-infectious inflammatory diseases such as psoriasis, ulcerative colitis or vasculitis caused by immune complexes [Bouchon et al, Nature 410:1103-1107 (2001)]. Moreover, when TREM-1 is bound to its ligand, there is a synergistic effect of LPS and an amplified synthesis of the pro-inflammatory cytokines TNF-α and GM-CSF, together with an inhibition of IL-10 production [Bleharski et al. J. Immunol. 170:3812-3818 (2003)]. In a murine model of LPS-induced septic shock, blockade of TREM-1 signalling protected the animals from death, further highlighting the crucial role of this molecule [Colonna et al, J. Infect. Dis. 187 (Suppl):S297-301 (2003), Bouchon et al, Nature 410:1103-1107 (2001)].

Studies demonstrate that TREM-1 plays a critical role in the inflammatory response to infection [Bouchon et al. J. Immunol. 164:4991-4995 (2000)]. Expression of TREM-1 is increased on myeloid cells in response to both bacterial and fungal infections in humans. Similarly, in mice the induction of shock by lipopolysaccharide (LPS) is associated with increased expression of TREM-1. Further, treatment of mice with a soluble TREM-1/Ig fusion protein, as a ‘decoy’ receptor, protects mice from death due to LPS or E. coli.

In 1991, the American College of Chest Physicians and the American Society of Critical Care Medicine published definitions for systemic inflammatory response syndrome (SIRS) and sepsis, with the aim of clarifying the diagnosis and treatment of these conditions and to aid interpretation of research in this field (see Table 1).

TABLE 1
Definitions for the systemic inflammatory response
syndrome (SIRS) and sepsis

| | |
|---|---|
| SIRS: Two or more of: | 1. Temperature >38° C. or <36° C. |
| | 2. Tachycardia >90 beats/minute |
| | 3. Respiratory rate >20 breaths/minute or |
| | PaCO₂ <4.3 kPa |
| | 4. White blood count >12 × 10⁹/l or <4 × 10⁹/l or |
| | >10% immature (band) forms |
| Sepsis: | SIRS due to infection |
| Severe sepsis: | Sepsis with evidence of organ hypoperfusion |
| Septic shock: | Severe sepsis with hypotension (systolic BP <90 |
| | mmHg) despite adequate fluid resuscitation or the |
| | requirement for vasopressors/inotropes to |
| | maintain blood pressure |

A pattern of physiological variables have been shown in critically ill patients in response to a range of insults including: trauma, burns, pancreatitis and infection. These include inflammatory responses, leucocytosis or severe leucopaenia, hyperthermia or hypothermia, tachycardia and tachypnoea and have been collectively termed the systemic inflammatory response syndrome (SIRS). This definition emphasises the importance of the inflammatory process in these conditions regardless of the presence of infection. The term “sepsis” is reserved for SIRS when infection is suspected or proven.

Sepsis is further stratified into severe sepsis when there is evidence of organ hypoperfusion, made evident by signs of organ dysfunction such as hypoxaemia, oliguria, lactic acidosis or altered cerebral function. Septic shock is severe sepsis complicated by hypotension defined as systolic blood pressure less than 90 mmHg despite adequate fluid resuscitation. Sepsis and SIRS may be complicated by the failure of two or more organs, termed multiple organ failure (MOF), due to disordered organ perfusion and oxygenation. In addition to systemic effects of infection, a systemic inflammatory response may occur in severe inflammatory conditions such as pancreatitis and burns.

In the intensive care unit, gram-negative bacteria are implicated in 50 to 60% of sepsis with gram-positive bacteria accounting for a further 35 to 40% of cases. The remainder of cases are due to the less common causes of fungi, viruses and protozoa.

Although there has been considerable interest in the TREM-1 receptor and its roles in inflammation and sepsis, there has hitherto been no identification of any biological ligand for the TREM-1 receptor.

The present inventors have now made a major breakthrough.

They have identified a ligand for the TREM-1 receptor and have confirmed that it is expressed upon neutrophils and monocytes from septic patients.

According to one embodiment of the present invention there is provided a screening method comprising providing a TREM-1 ligand or a derivative thereof and determining whether or not a test compound affects:
a) the binding of the ligand or derivative thereof to a TREM-1 receptor, or to a derivative thereof that comprises a TREM-1 ligand binding region

and/or
b) an activity that is modulated by the binding of a TREM-1 ligand to a TREM-1 receptor.

The method is preferably used for screening for compounds that are useful in the treatment of TREM-1 related inflammatory disorders, particularly sepsis and Inflammatory Bowel Disease (IBD).

Thus the method can be used for screening for drugs/drug candidates.

Here the method desirably comprises the step of determining whether or not a test compound blocks or reduces the binding of the TREM-1 ligand/derivative thereof to the TREM-1 receptor/derivative or whether or not it blocks or reduces an activity that is mediated by said binding.

If so, then the compound is concluded to be potentially useful in the treatment of TREM-1 related inflammatory disorders, particularly sepsis and Inflammatory Bowel Disease (IBD).

Preferably the method is used for screening for compounds useful in the treatment of sepsis mediated by a pathogen. The term “pathogen” is used herein to describe any infectious organism that can be detrimental to the health of a human or non-human animal host.

As discussed later, the present inventors have shown that the TREM-1 ligand is a useful marker of pathogen-mediated sepsis and can be used to distinguish this from SIRS conditions where there is no pathogenic involvement.

For example, sepsis may be due to a microbial infection.

The infection may for example be bacterial, fungal, protozoal or viral.

More preferably however it is bacterial or fungal.

Suitably the method uses cells that express a TREM-1 receptor or at least a ligand-binding part of a TREM-1 receptor.

(If desired, the intracellular part of a TREM-1 receptor may be replaced with a heterologous moiety that is normally not associated with the TREM-1 receptor. This is useful in certain reporter based screening systems. For example the cytoplasmic region of CD3ζ may be used, as discussed later in Example 11.)

The cells may be those that express the receptor naturally. Thus they may be neutrophils or monocytes. Such cells can be obtained from patients with sepsis. Alternatively, the cells need not be neutrophils or monocytes, but may be other cells that that do not normally express the TREM-1 receptor, but have been modified to express the TREM-1 receptor or a TREM-1 ligand binding part thereof. Modification may be performed by techniques known in the art. For example, the cells may be transfected with a vector encoding the TREM-1 receptor or at least the ligand binding part of this receptor and a suitable promoter (e.g. an inducible or constitutive promoter).

Thus the cells may be heterologous cells, relative to cells in which the receptor is normally expressed.

It is not however even essential for the TREM-1 receptor/ligand binding part thereof to be associated with a cell.

Soluble forms may be used. Multimeric forms may even be used.

For example, a tetramer comprising four soluble forms linked to a streptavidin scaffold may be used, as described in greater detail later on herein. Alternatively, a soluble form comprising the TREM-1 receptor extracellular domain fused to IgG contant regions may be used. Such a construct is described in Example 1 of WO/2004/081233. (The full content of WO/2004/081233 is hereby incorporated by reference.)

It is also possible to provide the receptor in an immobilised form, e.g. via an affinity column.

All of the above forms can be considered as derivatives of the receptor, provided that they still retain an ability to bind the TREM-1 ligand

Binding may be assessed quantitatively or qualitatively.

Thus, for example, the method may comprise determining the difference in binding of the TREM-1 ligand or derivative to the TREM-1 receptor or derivative in the absence of the test compound with that occurring in the presence of the test compound.

Alternatively a qualitative assay may simply determine whether or not binding has occurred.

Techniques for analysing binding are well known in the art. For example, the binding may be detected through use of a competitive immunoassay, a non-competitive assay system using techniques such as western blots, a radioimmunoassay, an ELISA (enzyme linked immunosorbent assay), a “sandwich” immunoassay, an immunoprecipitation assay, a precipitin reaction, a gel diffusion precipitin reaction, an immunodiffusion assay, an agglutination assay, a complement fixation assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarisation assay, a scintillation proximity assay, a homogeneous time resolved fluorescence assay, an IAsys analysis, or a BIAcore analysis.

Suitable techniques use a detectable label and measure changes in the amount of label detected.

The present inventors have identified CD177 (sometimes known as NB1 or PRV-1) as a TREM-1 ligand.

They have also shown that a monoclonal antibody to CD177 blocks the binding of constructs comprising the TREM-1 ligand to septic neutrophils expressing the TREM-1 receptor.

CD177 was well known prior to the present invention, but there was nothing to indicate that it was a TREM-1 ligand. Indeed there was no discussion at all of CD177 in connection with TREM-1 ligands.

CD177 is discussed in connection with autoimmune disorders. For example it is explained in Stroneck et al in Transl Med. 2004; 2: 8, that CD177is a neutrophil membrane glycoprotein that was first described by Lalezari et al while investigating a case of neonatal alloimmune neutropenia [Lalezari P, Murphy G B, Allen F H Jr. NB1, a new neutrophil-specific antigen involved in the pathogenesis of neonatal neutropenia J Clin Invest. 1971; 50:1108-1115)]. Occasionally, during pregnancy, a mother produces alloantibodies to neutrophil antigens than cross the placenta, react with neutrophils in the fetus, and cause the neonate to become neutropenic. One antigen recognized by such antibodies was described as “NB1” by Lalezari et al. Later, this antigen was renamed as Human Neutrophil Antigen-2a (HNA-2a) and the gp carrying this antigen was called NB1 gp [Bux J, Bierling P, von dem Borne A E G Kr, et al. ISBT Granulocyte Antigen Working Party. Nomenclature of Granulocyte Alloantigens. Vox Sang. 1999; 77:251].

In 2001 Kissel and colleagues sequenced the gene encoding NB1 gp and called the gene NB1 [Kissel K, Santoso S, Hofmann C, Stroncek D, Bux J. Molecular basis of the neutrophil glycoprotein NB1 (CD177) involved in the pathogenesis of immune neutropenias and transfusion reactions. European Journal of Immunology. 2001; 31:1301-1309]. However, this gene was highly homologous to a gene called PRV-1 that had been sequenced the year before. Temerinac and colleagues identified and sequenced PRV-1 in 2000 while searching for genes overexpressed in neutrophils from patients with polycythemia vera [Temerinac S, Klippel S, Strunck E, Roder S, Lubbert M, Lange W, Azemar M, Meinhardt G, Schaefer H E, Pahl H L. Cloning of PRV-1, a novel member of the uPAR receptor superfamily, which is overexpressed in polycythemia rubra vera. Blood. 2000; 95:2569-2576]. The coding regions of NB1 and PRV-1 differ at only 4 nucleotides that result in amino acid changes and Caruccio, Bettinotti, and colleagues have shown that PRV-1 and NB1 are alleles of a single gene [Bettinotti M P, Olsen A, Stroncek D. The Use of Bioinformatics to Identify the Genomic Structure of the Gene that Encodes Neutrophil Antigen NB1, CD177. Clinical Immunology. 2002; 102:138-144; Caruccio L, Walkovich K, Bettinotti M, Schuller R, Stroncek D. CD177 polymorphisms: correlation between high frequency single nucleotide polymorphisms and neutrophil surface protein expression. Transfusion. 2004; 44:77-82]. PRV-1 and NB1 are now considered to be alleles of the same gene, with PRV-1 being the more common allele in a normal population [Caruccio L, Bettinotti M, Fraser E, Director-Myska A, Arthur D C, Stroncek D F Blood. 2003;102:661a].

As indicated earlier, derivatives of CD177 can be used in the present invention.

The term “derivative” includes variants, fragments and fusion proteins.

Suitable derivatives bind to a TREM-1 receptor under physiological conditions. More suitably, they do not bind to any other cell surface protein present in vivo upon neutrophils or monocytes, especially to neutrophils or monocytes from septic patients. This enables them to be used in cell-based binding assays that are highly specific.

Most suitably, derivatives are specific for a TREM-1 receptor in the sense that they do not bind to any other protein that is normally found in the species (e.g. Homo sapiens) from which the receptor is obtained.

Variants of CD177 include allelic variants. Allelic variant may be intra-species or inter-species allelic variants. Suitable variants occur in mammals. More suitably they occur in rodents (e.g. mice, rats) or rabbits or in humans.

Non-allelic variants are also included. Such molecules can be prepared using recombinant DNA technology, automated synthesis, site directed mutagenesis, etc. Such techniques are now well developed.

Suitable variants have an amino acid sequence (or at least a part thereof) that is at least about 60%, 70%, 75%, 80%, 85%, 90%, 95%, or 98% identical to the amino acid sequence shown in FIG. 18, or at least to a part of the FIG. 18 sequence that is required for binding to the TREM-1 receptor for CD177.

This part is expected to be within a fragment corresponding to amino acids from 22 to 437 especially from 22 to 408 shown in FIG. 18. Thus this stretch of amino acids or (even a smaller part thereof that still binds to the CD177 receptor) can be used in the present invention and can also be used for sequence comparisons. The amino acid sequence 1 to 22 shown in FIG. 18 will not normally be present in the mature protein. Amino acid 408 is the amino acid used for attachment to the GPI anchor. An enzyme that cleaves the GPI anchor (e.g. Phospolipase C) can be use to release the 22 to 408 fragment as a soluble form. Other soluble forms are also possible and can be made by genetic engineering, as discussed in greater detail later on.

In order to determine the percentage sequence identity of two peptides/amino acid sequences or of two nucleic acid sequences, the sequences are aligned for optimal comparison purposes (e.g., gaps may optionally be introduced in one or both of a first and a second amino acid or nucleic acid sequence for optimal alignment and non-homologous sequences can be disregarded for comparison purposes).

For example, the length of a reference sequence aligned for comparison purposes is at least 30%, suitably at least 40%, more suitably at least 50%, even more suitably at least 60%, and even more suitably at least 70%, 80%, or 90% of the length of the reference sequence, such as the whole of the length of the reference sequence (e.g. when aligning a second sequence to the first amino acid sequence which has for example 100 amino acid residues, at least 30, suitably at least 40, more suitably at least 50, even more suitably at least 60, and even more suitably at least 70, 80 or 90 amino acid residues are aligned, such as 100 amino acid residues). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percentage identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

In one embodiment, the percentage identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4, and a length weight of 1, 2, 3, 4, 5, or 6.

In another embodiment, the percent identity between two nucleotide sequences is determined using the GAP program in the GCG software package (available at http://www.gcg.com), using a NWSgapdna.CMP matrix and a gap weight of 40, 50, 60, 70, or 80, and a length weight of 1, 2, 3, 4, 5, or 6. In another embodiment, the percentage identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12, and a gap penalty of 4. The nucleic acid and protein sequences of the present invention can further be used as a “query sequence” to perform a search against public databases to identify, for example, other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to NIP2b, NIP2cL, and NIP2cS nucleic acid molecules of the invention. BLAST protein searches can be performed with the XBLAST program, score=50, wordlength=3 to obtain amino acid sequences homologous to NIP2b, NIP2cL, and NIP2cS protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See http://www.ncbi.nlm.nih.gov.)

It is of course entirely reasonable to cover a wide range of variants for use in the present invention, because the skilled person appreciates that various changes can often be made to the amino acid sequence of a polypeptide with a desired property (such as binding to a receptor) whilst still retaining that property.

Such changes are summarised in sections (i) to (iv) below:

(i) Substitutions

The skilled person is aware that various amino acids often have similar properties so that they can often be interchanged without eliminating a desired property of that polypeptide (such as maintaining at least 20%, suitably at least 50%, more suitably at least 75% of the desired activity).

For example, the amino acids glycine, alanine, valine, leucine and isoleucine can often be substituted for one another (amino acids having aliphatic side chains). Of these possible substitutions it is most typical that glycine and alanine are used to substitute for one another (they have relatively short side chains) and that valine, leucine and isoleucine are used to substitute for one another (they have larger aliphatic side chains which are hydrophobic).

Other amino acids that can often be substituted for one another typically include:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains);
and cysteine and methionine (amino acids having sulphur containing side chains).

Substitutions of this nature are often referred to as “conservative” or “semi-conservative” amino acid substitutions.

Suitably the variant may contain 10 or fewer substitutions (e.g. 5 or fewer, more suitably 1 or 2).

(ii) Deletions

Deletions of inessential or undesired parts of a polypeptide can be made. This can be useful in reducing the size of a polypeptide. As discussed later, deletions can also be useful in producing soluble polypeptides if a polypeptide is normally membrane bound.

Suitably, the variant may contain one or two deletions, each of which is 20% or less (such as 10% or less) of the length of the reference sequence.

(iii) Insertions

Amino acid insertions can also be made. This may be done to alter the properties of the polypeptide (e.g. to assist in identification, purification or expression).

Suitably, the variant may contain one or two insertions, each of which is each of which is 20% or less (such as 10% or less) of the length of the reference sequence.

Polypeptides incorporating amino acid changes (whether substitutions, deletions and/or insertions) relative to a given sequence can be provided using any suitable techniques. For example, a nucleic acid sequence incorporating a sequence change can be provided by site directed mutagenesis. This can then be used to allow the expression of a polypeptide having a corresponding change in its amino acid sequence.

(iv) Combinations of the Above

One or more deletions, insertions and/or substitutions may of course be combined.

Other variants are also included. For example polypeptides comprising one or more amino acid analogues (including non-naturally occurring amino acids) may be used. Thus the present inventions includes mimetopes and peptidomimetics. The terms “mimetope” and “peptidomimetic” are used interchangeably herein. A “mimetope” of a compound X refers to a compound in which chemical structures of X necessary for functional activity of X have been replaced with other chemical structures which mimic the conformation of X. Examples of peptidomimetics include peptidic compounds in which the peptide backbone is substituted with one or more benzodiazepine molecules (see e.g., James, G. L. et al. (1993) Science 260:1937-1942) and “retro-inverso” peptides (see U.S. Pat. No. 4,522,752 to Sisto). The terms “mimetope” and “peptidomimetic” also refer to a moiety, other than a naturally occurring amino acid, that conformationally and functionally serves as a substitute for a particular amino acid in a peptide-containing compound without adversely interfering to a significant extent with the function of the peptide. Examples of amino acid mimetics include D-amino acids. Peptides substituted with one or more D-amino acids may be made using well known peptide synthesis procedures. Additional substitutions include amino acid analogues having variant side chains with functional groups, for example, b-cyanoalanine, canavanine, djenkolic acid, norleucine, 3-phosphoserine, homoserine, dihydroxyphenylalanine, 5-hydroxytryptophan, 1-methylhistidine, or 3-methylhistidine. Methods for preparing mimetopes and peptidomimetics are known in the art.

Turning now to fragments, as indicated earlier, fragments may be utilised in the screening methods. They may also be used for other purposes e.g. for raising antibodies, for binding studies; for therapeutic purposes (as discussed later), etc.

Fragments suitably include at least 10, 20, 30, 40, 50, 60, 70, 80, 90 or 100 amino acids of the amino acid sequence shown in FIG. 18 or of variants thereof.

Suitable fragments are soluble forms. The term “soluble” is used herein to distinguish from a polypeptide that is membrane-bound.

Generally the signal sequence (shown in italics, amino acids 1-22, in FIG. 18) will be absent.

A soluble form can be made by cleaving a GPI anchored protein with a suitable enzyme, as discussed earlier. Alternatively, genetic engineering techniques may be used to provide a protein that is secreted and does not have a GPI anchor.

Different lengths of soluble forms can be provided and can be derived from the extracellular portion of a TREM-1 ligand or variant.

Suitably, however, at least a TREM-1 receptor binding portion is present, as can be easily determined by binding studies.

If desired, the variants or fragments described above may be linked with heterologous moieties (i.e. moieties with which they are not normally linked in nature). Suitably the link is via a covalent bond, although non-covalent linkages are also within the scope of the present invention.

Thus, for example, fusion proteins may be provided.

The present invention encompasses fusion proteins in which the TREM-1 ligands or derivatives thereof (especially fragments) are recombinantly fused or chemically conjugated (including both covalent and non-covalent conjugations) to heterologous polypeptides (i.e., an unrelated polypeptide or portion thereof, suitably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences.

In one example, fusion is to sequences derived from various types of immunoglobulins. For example fusion can be to a constant region (e.g., hinge, CH2, and CH3 domains) of human IgG1 or IgM molecule, (for example, as described by Hudson & Souriauso (2003) Nature Medicine 9(1):129-134) so as to make the fused polypeptides or fragments thereof more soluble and stable in vivo. The short half-life of antibody fragments can also be extended by ‘pegylation’, that is, a fusion to polyethylene glycol (see Leong, S. R. et al. (2001) Cytokine 16:106-119). In one example of such fusions, described in WO 01/83525, Fc domains are fused with biologically active peptides. A pharmacologically active compound is produced by covalently linking an Fc domain to at least one amino acid of a selected peptide. Linkage to the vehicle increases the half-life of the peptide, which otherwise could be quickly degraded in vivo

Alternatively, fusion proteins comprising non-classical alternative protein scaffolds can be made (for example see Nygren & Skerra (2004) J Immunol Methods 290(1-2):3-28 or WO03049684).

Such fusion proteins can be used as an immunogen for the production of specific antibodies which recognize the polypeptides of the invention or fragments thereof.

In one particular embodiment, fusion proteins can be administered to a subject so as to inhibit interactions between the TREM-1 ligand and its receptor in vivo.

N-terminal signal sequence fusions to signal sequences can be provided if desired. Various signal sequences are commercially available. For example, the secretory sequences of melittin and human placental alkaline phosphatase (Stratagene; La Jolla, Calif.) are available as eukaryotic heterologous signal sequences. As examples of prokaryotic heterologous signal sequences, the phoA secretory signal (Sambrook, et al., supra; and Current Protocols in Molecular Biology, 1992, Ausubel, et al., eds., John Wiley & Sons) and the protein A secretory signal (Pharmacia Biotech; Piscataway, N.J.) can be listed. Another example is the gp67 secretory sequence of the baculovirus envelope protein (Current Protocols in Molecular Biology, 1992, Ausubel, et al., eds., John Wiley & Sons).

In another embodiment, fusion is to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz, et al., 1989, Proc. Natl. Acad. Sci. USA 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson, et al., 1984, Cell 37:767) and the “flag” tag (Knappik, et al., 1994, Biotechniques 17(4):754-761). These tags are especially useful for purification of recombinantly produced polypeptides.

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, 1992, Ausubel, et al., eds., John Wiley & Sons). The nucleotide sequence coding for a fusion protein can be inserted into an appropriate expression vector.

Suitable fusions proteins are multivalent in the sense that they have a plurality of parts (or ligands) that can bind to a TREM-1 receptor.

Thus for example a plurality of soluble CD177 ligands (i.e. proteins capable of specifically binding to CD177) may be joined together.

This can be achieved for example by linking soluble forms to a multivalent scaffold. Molecules such as immunoglobulins can be used to provide convenient multimeric scaffolds.

A dimer based upon fusing a TREM-1 ligand coding region with a region encoding a mutated form of the Fc portion of IgG is discussed in the examples (the Fc portion is modified so that it does not bind to Fc receptors). The dimer is produced upon association of the Fc regions once the fusion proteins polypeptides have been secreted into a cell culture medium.

It is not however essential to use an immunoglobulin-derived scaffold. The scaffold may be provided by any desired structure.

For example streptavidin can be used. As discussed earlier this has been successfully by the present inventors as a scaffold to provide a tetramer of the TREM-1 receptor. A similar technique can be used to provide a tetramer for the TREM-1 ligand or derivative thereof.

Different multimers (e.g. dimers, trimers, tetramers, heptamers, hexamers, etc.) may be provided by linking different numbers of TREM-1 ligands/derivatives thereof to an appropriate scaffold.

Whatever type of the scaffold is used, it is desired that it does not substantially interfere with the binding to the TREM-1 receptor.

Suitable structures are at least as capable of binding to the TREM-1 receptor as is the wild-type TREM-1 ligand. More suitably they have a higher probability of binding to the TREM-1 receptor.

If the structure is to be used in therapy, it is desired that the scaffold does not significantly increase inflammation in a mammalian (suitably human) host. Proteins that are already present in a given species can be used as the basis for suitable scaffolds, given that these are less likely to provoke immune responses.

It will be appreciated from the foregoing description that various fusion proteins can be used in the present invention.

A TREM-1 ligand, fragment or variant need not however be linked to another polypeptide, as in the case of a fusion protein, but can be linked to a surface.

This allows immobilisation upon the surface. For example plates, chips, columns, beads, matrixes, membrane, wells, etc. are often used to provide surfaces for immobilisation. Linkers can be used to attach the ligand, fragment or variant to the surface, as is well known in the art of immobilisation.

Immobilised forms may be used for many purposes, including purification, diagnosis, screening (especially high throughput screening), characterisation, storage, ease of handling, etc.

It will be appreciated from the foregoing description that many types of TREM-1 ligand or derivatives thereof can be used in the present invention, including variants, fragments, fusion proteins, etc.

The ligand or derivative may be provided in “isolated” form.

For the purposes of the present invention, an “isolated” polypeptide is considered to be substantially free of cellular material or other contaminating polypeptides from the cell or tissue source from which the protein is derived, or is substantially free of chemical precursors or other chemicals when chemically synthesised.

The language “substantially free of cellular material” includes preparations of a polypeptide in which the polypeptide is separated from cellular components of the cells from which it is isolated or recombinantly produced. Thus, a polypeptide/protein that is substantially free of cellular material includes preparations of the polypeptide/protein having less than about 50%, 40%, 30%, 20%, 10%, 5%, 2.5%, or 1%, (by dry weight) of contaminating protein.

When the polypeptide is recombinantly produced, it is also suitably substantially free of culture medium, i.e. the culture medium represents less than about 50%, 40%, 30%, 20%, 10%, or 5% of the volume of the protein preparation. When the polypeptide is produced by chemical synthesis, it is suitably substantially free of chemical precursors or other chemicals, i.e., it is separated from chemical precursors or other chemicals which are involved in the synthesis of the protein. Accordingly, such preparations of the polypeptide/protein have less than about 50%, 40%, 30%, 20%, 10%, or 5% (by dry weight) of chemical precursors or compounds other than polypeptide fragment of interest.

Of course other substances for use in the present invention) may also be provided in isolated form. Isolated forms may be used for analysis of structure/function, for binding studies, for screening, for raising or selecting antibodies, etc. The fact that they there is relatively little or no contamination with other proteins means that results are unlikely to be adversely affected by contamination.

Turning now to medical uses of the present invention, the invention includes the use of a compound that blocks or reduces the binding of a TREM-1 ligand to the TREM-1 receptor in the manufacture of a medicament for treating a disorder that is characterised by the release of one or more proinflammatory cytokines or chemokines.

The disorder may be any inflammatory disorder (or other disorder) that is mediated by the binding of the TREM-1 ligand to a TREM-1 receptor. Examples of inflammatory disorders include (but are not limited to) acute and chronic inflammatory disorders, sepsis, acute endotoxemia, encephalitis, Inflammatory Bowel Disease (IBD), Chronic Obstructive Pulmonary Disease (COPD), allergic inflammatory disorders, asthma, pulmonary fibrosis, pneumonia, Community acquired pneumonia (CAP), Ventilator associated pneumonia (VAP), Acute respiratory infection, Acute respiratory distress syndrome (ARDS), Infectious lung diseases, Pleural effusion, Peptic ulcer, Helicobacter pylori infection, hepatic granulomatosis, arthritis, rheumatoid arthritis, osteoarthritis, inflammatory osteolysis, ulcerative colitis, psoriasis, vasculitis, autoimmune disorders, thyroiditis, Meliodosis, (mesenteric) Ischemia reperfusion, Filovirus infection, Infection of the urinary tract, Bacterial meningitis, Salmonella enterica infection, Marburg and Ebola viruses infections.

Furthermore, TREM-1 signalling is implicated in diseases in which monocyte-platelet and neutrophil-platelet aggregates play an important role (Haselmayer et al. Blood 2007 110:1029-1035). For example, circulating leukocyte-platelet aggregates, especially monocyte-platelet aggregates, promote the formation of atherosclerotic lesions, are increased in acute coronary syndromes, stroke, and peripheral vascular disease, and are an early marker of acute myocardial infarction. Increased circulating monocyte-platelet and neutrophil-platelet aggregates have also been reported in numerous other conditions, including diabetes mellitus, cystic fibrosis, asthma, preeclampsia, placental insufficiency, migraine, nephrotic syndrome, hemodialysis, sickle cell disease, systemic inflammatory response syndrome, septic multiple organ dysfunction syndrome, antiphospholipid syndrome, systemic lupus erythematosus, rheumatoid arthritis, inflammatory bowel disease, myeloproliferative disorders, Kawasaki disease, and Alzheimer disease (Michelson and Newburger, Blood 2007 110:794-795)

Preferably however the disorder is sepsis and is mediated by a pathogen.

More preferably it is microbial mediated sepsis.

Most preferably it is sepsis mediated by fungi or bacteria.

An example of sepsis that is microbially mediated is pneumonia.

Alternatively, preferably the disorder is Inflammatory Bowel Disease.

The term “pneumonia” as defined herein, means, an inflammation of the lung caused by infection by extracellular pathogens such as bacterial infection, and non-bacterial infections (for example, infection by Blastomyces dermatitidis, Histoplasma capsulatum, Coccidioides, Sporothrix schenckii, Pneumocystis carinii, Cryptococcus, Aspergillus, or Mucor sp.), protozoal infections or parasitic infections (for example, those caused by Toxoplasma gondii, Strongyloides stercoralis, Ascaris, hookworm, Dirofilaria, Paragonimus, or Entamoeba histolytica) where increased expression of sTREM-1 can be detected.

Pneumonia includes “Lobar Pneumonia” (which occurs in one lobe of the lung) and Bronchopneumonia (tends to be irregularly located in the lung). Furthermore, pneumonia is often classified into two categories that may help predict the organisms that are the most likely culprits. “Community-acquired (pneumonia contracted outside the hospital). Pneumonia” in this setting often follows a viral respiratory infection. It affects nearly 4 million adults each year. It is likely to be caused by Streptococcus pneumoniae, the most common pneumonia-causing bacteria. Other organisms, such as atypical bacteria called Chlamydia or Mycoplasma pneumonia are also common causes of community-acquired pneumonia. “Hospital-acquired pneumonia” contracted within the hospital is often called nosocomial pneumonia. Hospital patients are particularly vulnerable to gram-negative bacteria and staphylococci.

A wide range of compounds can be used in treatment of the above-mentioned disorders.

One example of such a compound is an antibody. Suitably the antibody binds to the TREM-1 ligand (or variant, fragment or fusion proteins thereof as appropriate).

Most suitably it binds to a part of the TREM-1 ligand that is responsible for binding to the TREM-1 receptor.

The antibody may be monoclonal or polyclonal.

Polyclonal antibodies can be raised by stimulating their production in a suitable animal host (e.g. a mouse, rat, guinea pig, rabbit, sheep, goat or monkey) when a TREM-1 ligand or derivative as immunogen is injected into the animal. If necessary, an adjuvant may be administered together with the substance of the present invention. The antibodies can then be purified by virtue of their binding to a substance of the present invention.

For example the immunogen may be CD177 or a fragment or variant thereof. Alternatively the immunogen may be cells expressing a TREM-1 ligand, such as neutrophils expressing a TREM-1 ligand such as CD177. Most suitably the immunogen is of human type (eg human CD177 or human cells expressing TREM-1 ligand).

Monoclonal antibodies can be produced from hybridomas. These can be formed by fusing myeloma cells and spleen cells from animals which produce the desired antibody in order to form an immortal cell line. Thus the well-known Kohler & Milstein technique (Nature, 256, 52-55 (1975)) or variations upon this technique can be used.

Techniques for producing monoclonal and polyclonal antibodies which bind to a particular polypeptide are now well developed in the art. They are discussed in standard immunology textbooks, for example in Roitt et al, Immunology second edition (1989), Churchill Livingstone, London.

In addition to whole antibodies, the term “antibody” is used herein to include derivatives thereof which are capable of binding to polypeptides of the present invention. Thus the present invention includes antibody fragments and synthetic constructs. Examples of antibody fragments and synthetic constructs are given by Dougall et al in Tibtech 12 372-379 (September 1994).

Antibody fragments include, for example, Fab, F(ab′)₂ and Fv fragments. Fv fragments can be modified to produce a synthetic construct known as a single chain Fv (scFv) molecule. This includes a peptide linker covalently joining Vₕ and Vₗ regions, which contribute to the stability of the molecule. Other synthetic constructs which can be used include CDR peptides. These are synthetic peptides comprising antigen-binding determinants. Peptide mimetics may also be used. These molecules are usually conformationally restricted organic rings which mimic the structure of a CDR loop and which include antigen-interactive side chains.

Synthetic constructs include chimeric antibodies. Here one or more parts of an antibody are derived from one animal (usually rodent) and one or more parts from another animal (usually humans). In practice such antibodies are produced by recombinant technology methods whereby DNA encoding a desired fusion protein is cloned and inserted into a suitable expression system. Preferred expression systems are mammalian cell cultures (e.g. CHO cells)

Preferred chimeric antibodies are humanised antibodies, sometimes known as CDR grafted antibodies. These are alternatives to more traditional chimeric antibodies. Here only the complimentarity determining regions from non-human (usually rodent) antibody V-regions are combined with framework regions from human V-regions. These antibodies are considered to be less immunogenic than older style chimeric antibodies, where the whole of the variable regions are derived from non-human animals. Thus undesired side effects are less likely.

Completely human antibodies can also be produced. For ethical reasons it is not desirable to produce these directly from humans. However they can be made by a variety of methods known in the art including phage display using antibody libraries derived from human immunoglobulin sequences. (See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741.). Human antibodies can also be produced using transgenic mice (see Lonberg and Huszar (1995), Int. Rev. Immunol. 13:65-93). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598; which are incorporated by reference herein in their entireties. In addition, companies such as Abgenix, Inc. (Freemont, Calif.), Medarex (NJ) and Genpharm (San Jose, Calif.) can be engaged to provide human antibodies directed against a selected antigen using technology similar to that described above. Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988, Bio/technology 12:899-903).

It is of course possible to provide any of the aforesaid antibodies/constructs with an additional moiety which provides the molecule with some desirable property in addition to antigen binding. For example the moiety may be a detectable label, a compound that increases the stability/half life of the antibody, in vivo etc.

Modifications such as lipidation can be used to stabilize antibodies and to enhance uptake and tissue penetration (e.g., into the brain). A method for lipidation of antibodies is described by Cruikshank, et al., 1997, J. Acquired Immune Deficiency Syndromes and Human Retrovirology 14:193). Reference can also be made to Leong, S. R. et al. (2001) Cytokine 16:106-1. Here it is explained that the half-life of antibody fragments can also be extended by ‘pegylation’, that is, by fusion to polyethylene glycol.

As is clear from the foregoing discussions, a wide range of antibodies/constructs can be used to block or reduce binding of the TREM-1 ligand to a TREM-1 receptor.

Suitably however a monoclonal antibody is used (e.g. the antibody R33).

Aspects of the invention include: a method for obtaining anti-TREM-1 ligand antibodies comprising providing a TREM-1 ligand or a derivative thereof and using it to generate antibodies in a non-human host, e.g. by immunising said non-human host (eg rabbit or rodent, such as mouse or rat) with TREM-1 ligand or a derivative thereof as immunogen; also a method for obtaining anti-TREM-1 ligand antibodies comprising providing cells such as neutrophils, which present on their surface a TREM-1 ligand or a derivative thereof and using them to generate antibodies in a non-human host e.g. by immunising said non-human host (eg rabbit or rodent, such as mouse or rat) with cells such as neutrophils, which present on their surface a TREM-1 ligand or a derivative thereof as immunogen.

An alternative way of blocking or reducing binding of the TREM-1 ligand to a TREM-1 receptor is to use a soluble form of the TREM-1 ligand or a soluble variant thereof, e.g. a multimer as described earlier.

This can bind to a TREM-1 receptor so it is no longer physically available for binding to the naturally occurring membrane bound form ligand, or at least its availability is reduced. This can prevent or reduce the pro-inflammatory release of cytokines and chemokines.

It is also possible to block or reduce the expression of the TREM-1 ligand, rather than rely upon blocking the binding of the ligand to the receptor. This can be done by blocking or reducing transcription or by blocking or reducing translation.

Thus for example transcriptional blockers of the gene for the TREM-1 ligand or down-regulators may be provided.

Alternatively, the gene for the TREM-1 ligand may be inactivated (e.g. by using targeted homologous recombination techniques to disrupt the gene/promoter).

In a further alternative, antisense molecules may be provided. These may hybridise to TREM-1 RNA so as to prevent or reduce translation thereof. Suitably hybridisation is specific so that there is not significant hybridisation to different RNA molecules produced naturally by neutophils or monocytes in vivo.

Hybridisation can be tested in vitro if desired. Thus stringent conditions can be provided and it can then be determined whether or not hybridisation occurs. Suitable antisense molecules hybridise under stringent conditions.

One example of stringent hybridisation conditions involves using a pre-washing solution of 5×SSC, 0.5% SDS, 1.0 mM EDTA (pH 8.0) and attempting hybridisation overnight at 55° C. using 5×SSC. However, there are many other possibilities. Some of these are listed in Table 1 of WO98/45435, for example. (See especially the conditions set out under A-F of that table and, less suitably, those listed under G to L or M to R.) Hybridisation conditions are discussed in detail at pp 1.101-1.110 and 11.45-11.61 of Sambrook et al [Molecular Cloning, 2nd Edition, Cold Spring Harbor Laboratory Press (1989)].

Antisense molecules can be introduced via a suitable vehicle (e.g. a liposome). They may even be introduced directly e.g. by using gene gun technology. Alternatively a vector may be provided that produces such molecules in vivo.

As an alternative to antisense molecules, double stranded RNA molecules that partake in RNA interference (RNAi) may also be used. Here targeted RNA is physically cleaved and therefore the mechanism of action of RNAi is quite different from that of antsisense molecules that act simply by binding to RNA so that it is no longer available for translation. In 2006, Andrew Fire and Craig C. Mello shared the Nobel Prize in Physiology or Medicine for their work on RNA interference in the nematode worm C. elegans, [Fire A, Xu S, Montgomery M, Kostas S, Driver S, Mello C (1998). “Potent and specific genetic interference by double-stranded RNA in Caenorhabditis elegans”. Nature 391 (6669): 806-11.] Since then various authors have discussed practical applications of RNAi to reducing/blocking gene expression. Relevant papers include: Dorsett, Y and Tuschl, T (2004). siRNAs: Applications in functional genomics and potential as therapeutics. Nature Reviews 3, 318-329; Hannon, G J and Rose, J J (2004). Unlocking the potential of the human genome with RNA interference. Nature 431, 371-378; Soutschek, J et al. (2004). Therapeutic silencing of an endogenous gene by systematic administration of modified siRNAs. Nature 432, 173-178; Morrisey, D V et al. (2005). Potent and persistent in vivo anti-HBV activity of chemically modified siRNAs. Nat. Biotechnol. 23, 1002-1007; Palliser, D (2006). An siRNA-based microbicide protects mice from lethal herpes simplex virus infection. Nature 439, 89-94; and Zimmermann T S et al. (2006). RNAi-mediated gene silencing in non-human primates. Nature 441, 111-114.

Ribozymes may also be used. These are single stranded RNA molecules (usually with double stranded hairpin regions) that have enzymatic activity. Ribozymes can be engineered that bind to and cleave target RNA molecules. This is discussed for example by Citti and Rainaldi in Curr Gene Ther. 2005 February; 5(1):11-24 “Synthetic hammerhead ribozymes as therapeutic tools to control disease genes”.

It will be appreciated from the foregoing description that many different compounds can be used in respect of the medical uses of the present invention. Indeed, in addition to the compounds discussed above, compounds identified by the screening methods discussed earlier can also be used. (The term “compound” is used in a non-limiting manner and can be any biological or synthetic moiety that is suitable for the uses described herein.)

The compound may be administered as a pharmaceutical composition together with a pharmaceutically acceptable acceptable diluent, carrier or excipient.

As used herein the language “pharmaceutically acceptable diluent, carrier or excipient” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions containing a peptide or polypeptide of the invention. Such compositions can further include additional active agents. Thus, the invention further includes methods for preparing a pharmaceutical composition by formulating a pharmaceutically acceptable carrier with a peptide or polypeptide of the invention and one or more additional active compounds.

A pharmaceutical composition of the invention is formulated to be compatible with its intended route of administration. Examples of routes of administration include parenteral, e.g., intravenous, intradermal, subcutaneous, transdermal (topical), transmucosal, intra-articular, intraperitoneal, and intrapleural, as well as oral, inhalation, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy injectability with a syringe exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the more suitable methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavouring agent, such as peppermint, methyl salicylate, or orange flavouring.

For administration by inhalation, the compounds are delivered in the form of an aerosol spray from a pressurized container or dispenser which contains a suitable propellant, e.g. a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As defined herein, a therapeutically effective amount of a polypeptide (i.e., an effective dosage) suitably ranges from about 0.001 to 30 mg/kg body weight, suitably about 0.01 to 25 mg/kg body weight, more suitably about 0.1 to 20 mg/kg body weight, and even more suitably about 1 to 10 mg/kg, 2 to 9 mg/kg, 3 to 8 mg/kg, 4 to 7 mg/kg, or 5 to 6 mg/kg body weight.

For antibodies, a suitable dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible.

Of course the actual dosage will be determined by a physician. If desired, a low starting dosage can be used and can gradually be increased until a beneficial effect is obtained. If side effects develop, then the dosage can be reduced in accordance with normal clinical practice.

The pharmaceutical compositions can be included in a container, pack, or dispenser together with instructions for administration.

The present invention also has various diagnostic applications.

It includes method steps that may provide information useful in the diagnosis of a disorder characterised by the release of one or more pro-inflammatory cytokines or chemokines.

The disorder may be any of the disorders discussed earlier in respect of medical uses.

Thus, for example, the invention includes a method comprising obtaining a biological sample and analysing the sample for a TREM-1 ligand or for TREM-1 ligand mRNA.

The sample can be a sample of whole blood, blood serum, blood plasma, urine, a cellular fraction of blood, a tissue sample, etc.

The sample is suitably a sample comprising cells (suitably neutrophils and/or monocytes) if it is desired to analyse membrane bound TREM-1 ligand. Similarly cells will normally be used if it is desired to analyse mRNA

The sample is suitably a sample comprising extracellular fluid (e.g. serum, plasma or urine) if it is desired to analyse soluble TREM-1 ligand. This is because the soluble form is shed into extracellular fluid.

The sample will normally be taken from a patient thought to have, or to be at risk of having, any of the disorders discussed earlier.

The presence or absence of the ligand or of corresponding mRNA may simply be identified. This can be useful if this is not present at all in a healthy individual or is present only at very low levels that are difficult to detect.

Suitably however the method includes a step of quantifying the TREM-1 ligand or TREM-1 ligand mRNA in the biological sample.

It may further comprise a negative control comparing the level of the ligand or corresponding RNA with a control level or range corresponding to what would be expected for a healthy individual.

If the level of TREM-1 ligand or TREM-1 ligand mRNA is significantly above that of the control this may be an indicator that an individual is likely to have the disorder

It may comprise a positive control, comprising comparing the level of the ligand or corresponding mRNA with a control level or range corresponding to what would be expected from an individual having the disorder.

If the level of TREM-1 ligand or TREM-1 ligand mRNA is significantly close to that of the positive control this may then be an indication that an individual is likely to have the disorder

The method may for example use an antibody to the ligand.

(Now that the ligand has been identified, it can be used to generate antibodies by standard techniques, as discussed earlier.)

A suitable antibody is specific to the ligand. It may be a monoclonal antibody (e.g. R33).

If the method detects TREM-1 mRNA in a sample then a nucleic acid molecule that hybridises to the TREM-1 mRNA can be used (e.g. a probe or primer).

Alternatively, the mRNA may be used to generate cDNA and a nucleic acid molecule that hybridises to the cDNA may be used. If desired amplification techniques such as reverse PCR can be used, although these are not essential.

Suitably the nucleic acid molecule is capable of hybridising under stringent conditions, as described earlier.

A further method that is useful in diagnosis is to provide a soluble form of the TREM-1 ligand or soluble variant thereof to bind to the TREM-1 receptor. This can be used to detect the TREM-1 receptor and/or to quantify the amount of receptor present in a sample.

In addition to the methods described above, the present invention also provides diagnostic kits.

It provides a kit for diagnosing a disorder that is characterised by the release in vivo of one or more pro-inflammatory cytokines or chemokines, wherein the kit comprises a compound that binds to a TREM-1 ligand or to DNA or RNA encoding said ligand.

Alternatively, the kit may comprises a TREM-1 ligand or a variant thereof (suitably a soluble form) that binds to the TREM-1 receptor.

The compound may for example be an antibody that binds to the TREM-1 ligand or a nucleic acid that hybridises to TREM-1 RNA or DNA.

The kit suitably includes comprising means for detecting and/or quantifying the binding.

This means may for example be one or more indicators that provide a visible change if said disorder is present. The indicator(s) may for example provide a colour change or a change in marking.

The kit may itself include one or more controls. For example it may comprise controls comprising biological samples from healthy patients. The samples may comprise cells (e.g. neutrophils and/or monocytes), as discussed earlier.

Alternatively they may be cell free. For example serum, plasma or urine samples may be provided if it is desired to screen to look for soluble forms of the TREM-1 ligand.

The controls need not even be physical samples. They may simply be indicators of what would be expected for healthy patients. Such indicators can be provided on instructions, packaging, labelling, etc. They may be in the form of charts, figures, ranges, etc.

Components of the kit may be enclosed within different containers, which may be sealed and may be in sterile form. The containers may be within a package for the kit, along with instructions for determining whether a subject is at risk of developing a disorder as described previously.

In addition to the aforesaid kits, the invention also includes kits for identifying the presence of a mutant form of a TREM-1 ligand.

The term “mutant form” is used herein to distinguish from the most common form known in nature in a given species, which is usually known as the “wild type”. Thus in the case of a gene encoding a mutant form, this may will differ from a gene encoding the wild type form by one or more coding nucleotides. In the case of a polypeptide a mutant form may differ by one or more amino acids. Mutant forms therefore include allelic variants.

Such a kit may, for example, comprise an antibody that binds more strongly to a mutant form than to a wild type form of the ligand, or may comprise a nucleic acid that binds more strongly to a nucleic acid encoding the mutant form than to a nucleic acid encoding the wild type form.

If desired, the kit may comprise a control allowing binding to be compared with binding to the wild type ligand or to a nucleic acid encoding the wild type ligand.

The antibody may be specific for the mutant form of the TREM-1 ligand. The nucleic acid may be specific (under stringent hybridisation conditions) for a nucleic acid encoding the mutant form of the TREM-1 ligand.

Mutant forms are of interest because they can identify individuals that may be more or less prone to a particular disorder (especially the disorders discussed herein) than individuals with the wild type gene. They can also be useful in research, in cell or tissue typing, in forensics, in diagnosis, etc.

A further aspect of the present invention is that of non-human animal, e.g. for use as an animal model, that has reduced expression of TREM-1 and/or of a TREM-1 ligand, relative to the wild type animal. Preferably it has no expression of TREM-1 and/or of a TREM-1 ligand. A further aspect of the invention is a non-human animal which also has reduced expression of TREM-3 relative to the wild type animal, and preferably has no expression of TREM-3.

Such animals are useful as a control compared to the wild type animal. They can be used to analyse the effectiveness of substances identified by the screening methods described earlier. They can also be used to assess side effects.

The provision of suitable animal models can be useful in reducing the overall number of test animals needed for screening for side effects, for drug efficacy, etc. Thus, these models can be beneficial in reducing overall animal suffering.

Preferably, the non-human animal is a complete knock-out for the TREM-1 ligand or the TREM-1 receptor. Thus it does not produce functional TREM-1 ligand or functional TREM-1 receptor.

This can be done by using recombination techniques to delete or inactivate essential regions of the TREM-1 gene.

Breeding techniques can then be used to generate a line of mice that are homozygous for the modification.

In some cases transgenic animals may be provided have a plurality of genes knocked out relative to the wild type, especially TREM-3

For example TREM-1 TREM-3 double knock-out rodents, particularly mice (TREM-1-3−/− mice) may be provided, as discussed later.

### BACKGROUND AND EXAMPLES

The present invention will now be described by way of example only, with reference to the accompanying drawings, wherein:

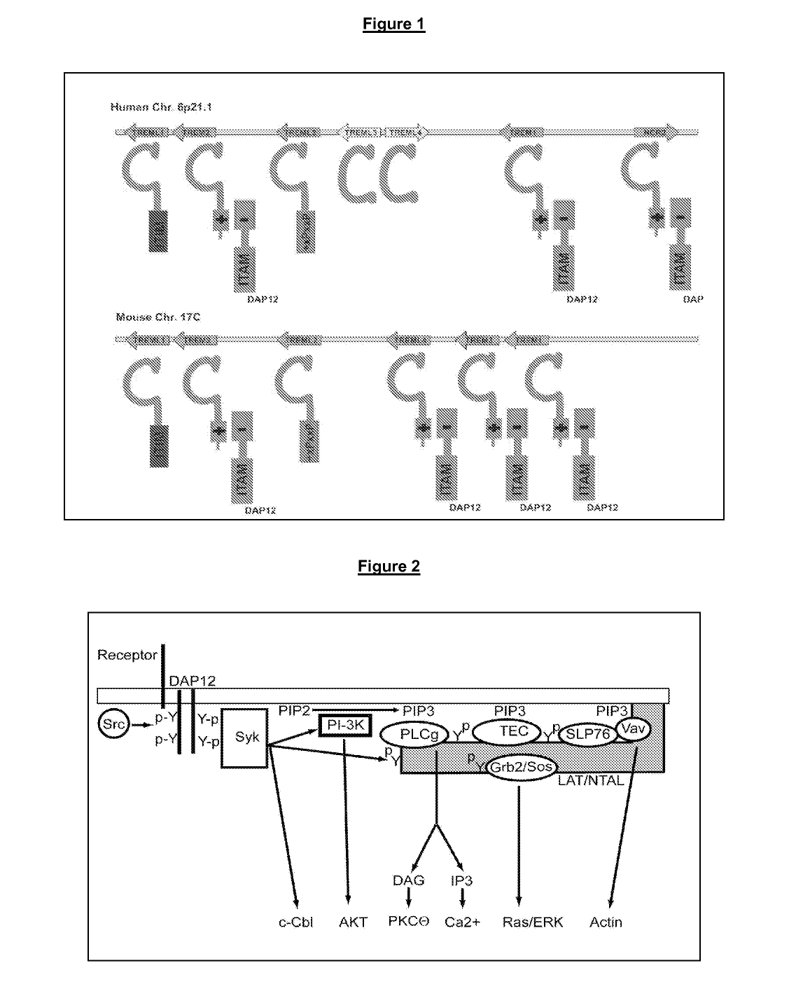

FIG. 1 (upper plate) shows the human and mouse TREM gene clusters. TREM gene clusters are located on human chromosome 6 p.21.1 and mouse chromosome 17C. Both clusters include genes encoding Trem1, Trem2, Treml1 (encodes TLT-1) and Treml2 (encodes TLT-2). Trem-1 and Trem-2 signal through the ITAM-containing adaptor DAP12. TLT1 contains a cytoplasmic ITIM for recruitment of cytosolic phosphatases. TLT-2 encodes a potential SH3 binding motif (+xPxxP, where + is arginine, x any amino acid, and P is proline). The human TREM cluster also includes Ncr2, which encodes the NK cell receptor NKp44, while no murine NKp44 homolog has been identified. It is not yet known whether two additional human genes, Treml3 and Treml4, encode functional proteins. The mouse TREM cluster includes genes encoding functional Trem-3 and Trem-L4 proteins. Trem3 is a pseudogene in human.

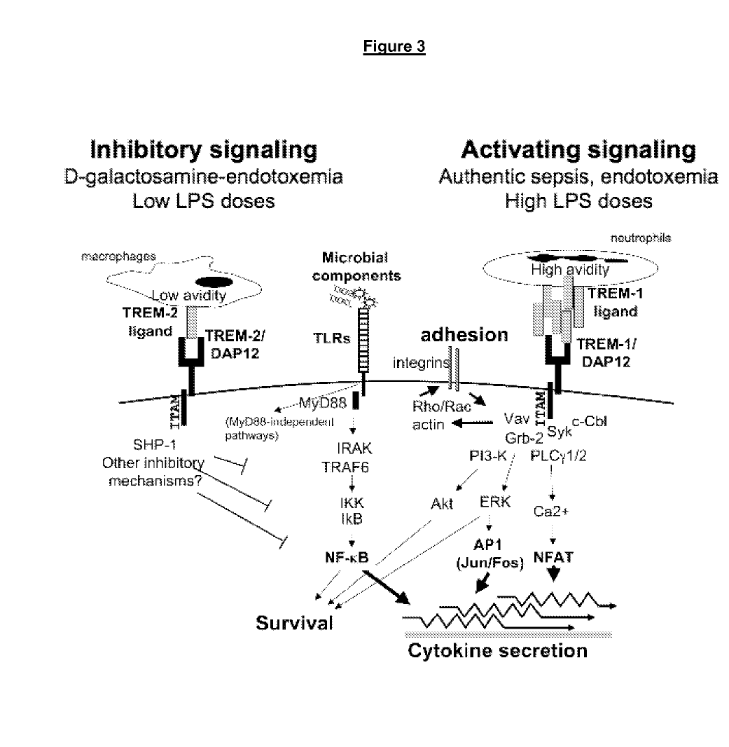

FIG. 2 (lower plate)_shows canonical DAP12 signalling. Analogous to other immunoreceptor tyrosine-based activation motif (ITAM)-containing adaptor proteins, crosslinking of receptors associated with DAP12 leads to phosphorylation of the tyrosines in the cytoplasmic ITAM motif by Src kinases. This leads to recruitment of SYK (or ZAP70) and subsequent phosphorylation of scaffolding molecules LAT and/or NTAL, and activation of PI-3K. LAT/NTAL recruit several effectors: PLCγ; TEC family members; the adaptor SLP76 in complex with Vav; the adaptor Grb2 in complex with Sos. PI-3K produces Ptdlns(3,4,5)P3 (PIP3) which contributes to recruitment of PLCγ, TEC, Vav to the cell membrane. All these intermediate signalling molecules lead to the recruitment/activation of Akt, c-CBL, and ERK, and to cytoskeletal remodelling (actin). PLCγ generates the secondary messengers DAG and IP3 leading to activation of PKCθ and calcium mobilization (Ca2+), respectively.

FIG. 3 compares activating vs. inhibitory signalling of DAP12. In this model we propose that sepsis or simple endotoxemia with high LPS doses (right) lead to multivalent engagement of TREM-1 on neutrophils by TREM-1 ligand, generating a signalling cascade that synergises with that of TLR at different levels. This results in increased cytokine secretion and, possibly, cell adhesion and cell survival. In contrast, nonseptic conditions such as D-galactosamine-potentiated endotoxemia induce low occupancy of TREM-2 on macrophages by TREM-2 ligand, resulting in partial phosphorylation of DAP12 and recruitment of phosphatase SHP-1 or other inhibitory molecules that reduce cell responsiveness to TLRs.

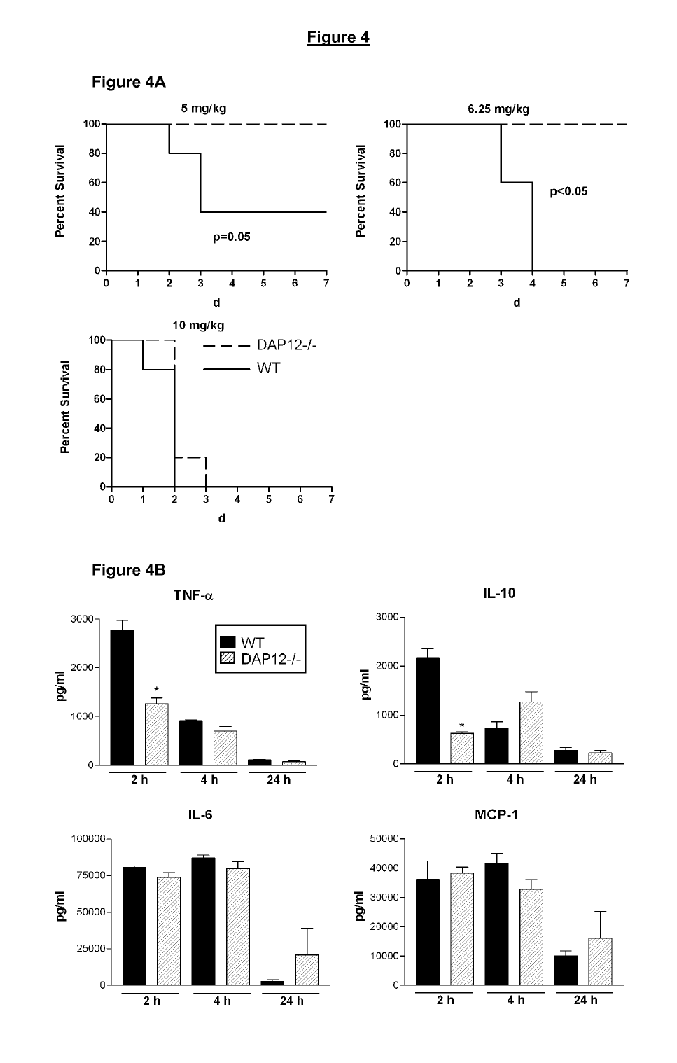

FIG. 4 shows that DAP12 signalling augments mortality and inflammatory cytokine levels during endotoxemia. (A) Survival of WT and DAP12−/− mice after endotoxemia was measured at three different doses, 5 mg/kg, 6.25 mg/kg, and 10 mg/kg. At both 5 and 6.25 mg/kg DAP12−/− mice had improved survival as compared to WT (p≦0.05 by Log-Rank Test). At 10 mg/kg both strains succumbed. (B) Plasma was harvested from WT and DAP12−/− mice 2, 4 or 24 hours after injection of 5 mg/kg LPS. At 2 hours, WT mice had increased levels of TNF-α and IL-10 (*=p<0.05 vs. WT by Mann-Whitney Test).

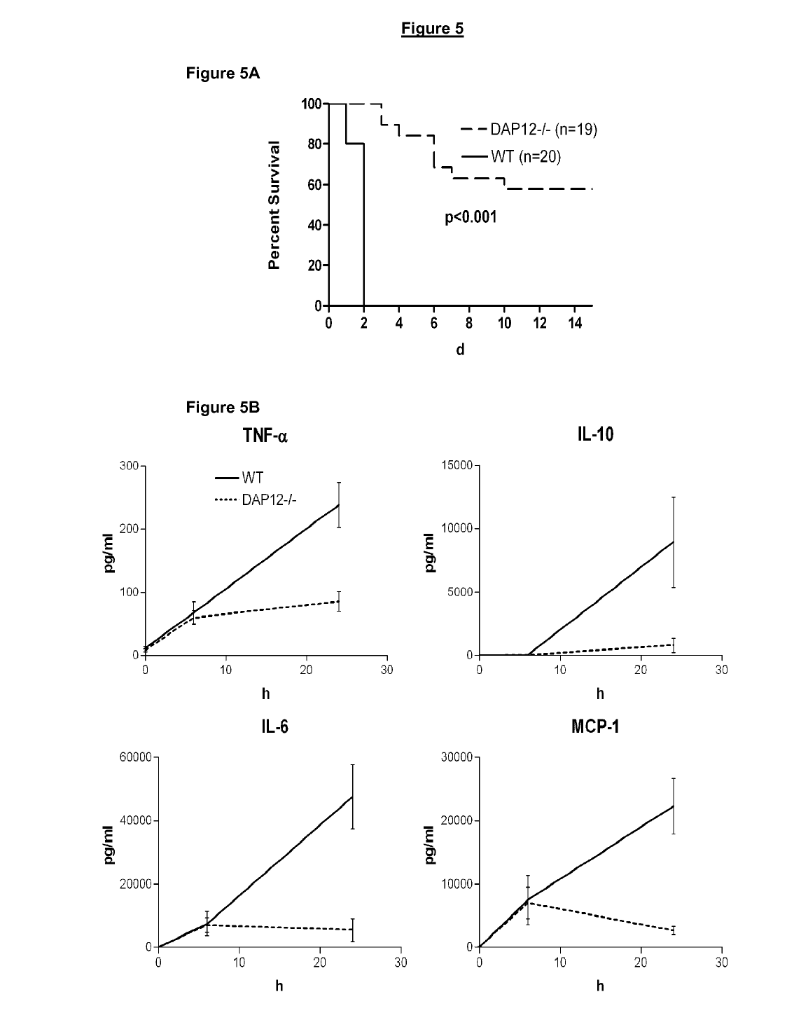

FIG. 5 shows that DAP12 signalling augments mortality and inflammatory events during bacterial sepsis WT and DAP12−/− mice were subjected to CLP and (A) survival and (B) cytokine production were assessed. DAP12−/− mice are resistant to CLP as compared to WT (p<0.001 by Log-Rank Test). Plasma was harvested from WT or DAP12−/− mice 6 or 24 hours after CLP and cytokine levels were measured. At 6 h we found equal levels of MCP-1, IL-6 and TNF-α in WT and DAP12−/− mice. By 24 h WT mice had significantly higher levels of MCP-1, IL-6, TNF-α and IL-10 (p<0.05 by Mann-Whitney Test).

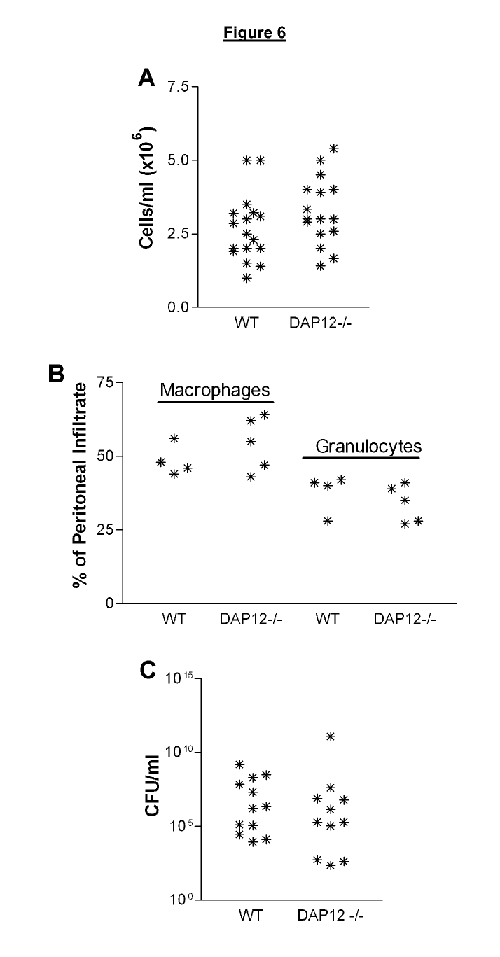

FIG. 6 shows that DAP 12 signalling does not contribute to cellular recruitment or bacteriocidal activity. 24 hours after CLP, peritoneal exudate was harvested by peritoneal lavage. Total cell numbers (A), distribution of cell types (B) and bacterial load (C) were measured. We found no difference between WT and DAP12−/− mice.

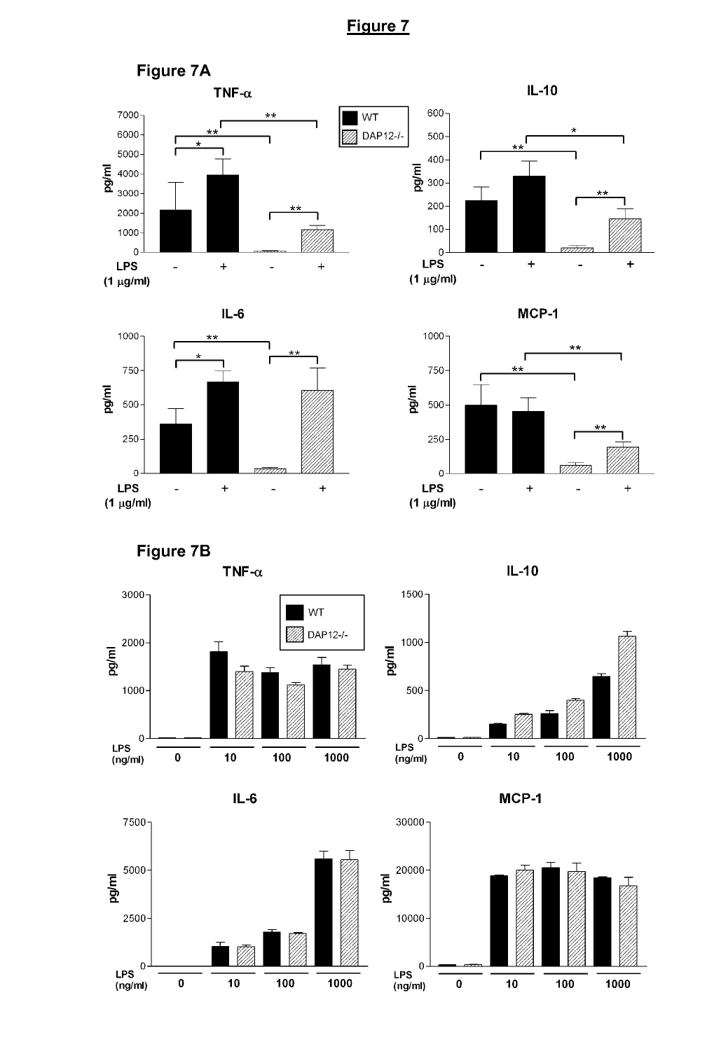

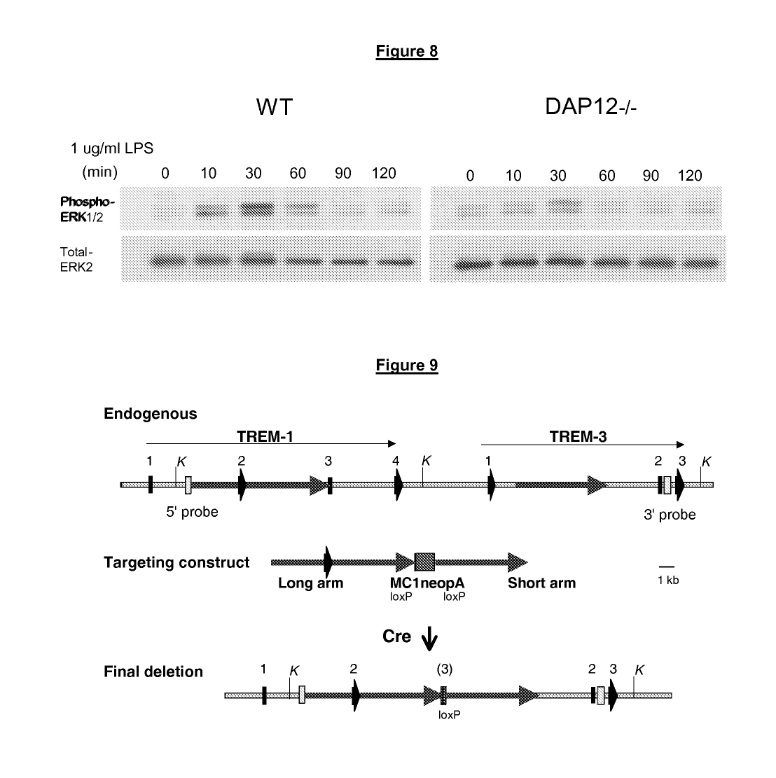

FIG. 7 shows that DAP12 augments production by macrophages after sepsis but not sterile peritonitis. (A) WT and DAP12−/− mice were subjected to CLP and peritoneal cells harvested after 24 hours. Cells were cultured ex-vivo with or without stimulation with LPS (1 μg/ml) and levels of cytokine in the supernatant were measured. With no stimulation, WT cells (solid bars) produced more IL-6, MCP-1, TNF-α and IL-10 as compared to DAP12−/− cells (hashed bars) (p<0.05 by Mann-Whitney Test). After LPS stimulation, WT cells produced increased amounts of TNF-α, MCP-1, and IL-10. (B) Cells were also harvested 72 hours after i.p. injection of thioglycollate broth and cultured ex-vivo with or without LPS (10, 100 or 1000 ng/ml) (B). There was no statistically significant difference between WT and DAP12−/− cells, although there was a trend toward increased IL-10 by the DAP12−/− cells with maximal stimulation.

FIG. 8 shows ERK phosphorylation after stimulation of peritoneal exudates cells (PES) with LPS in vitro. 24 hrs after CLP, PEC were harvested, stimulated with LPS for various time points, and cell lysates were resolved by SDS-PAGE and immunoblotted for phospho-ERK1/2. Total ERK was determined as loading control. WT mice showed significant more phosphorylation than DAP12−/− mice after 30 minutes of stimulation.

FIG. 9 illustrates the generation of TREM-1/TREM-3 deficient mice

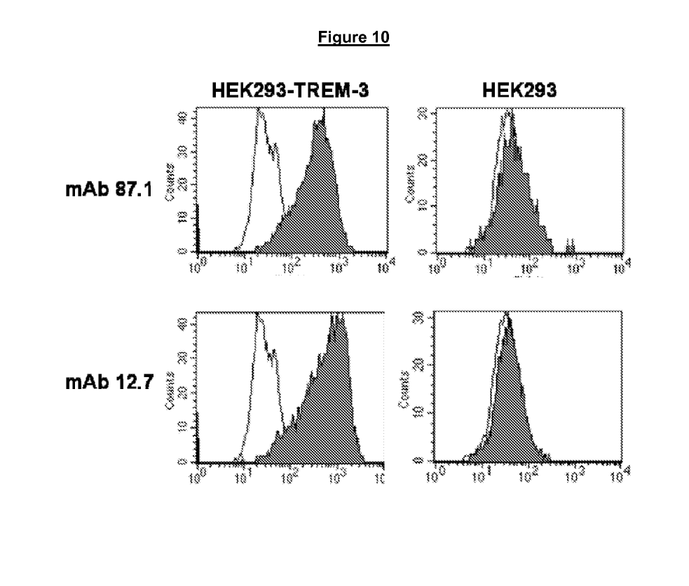

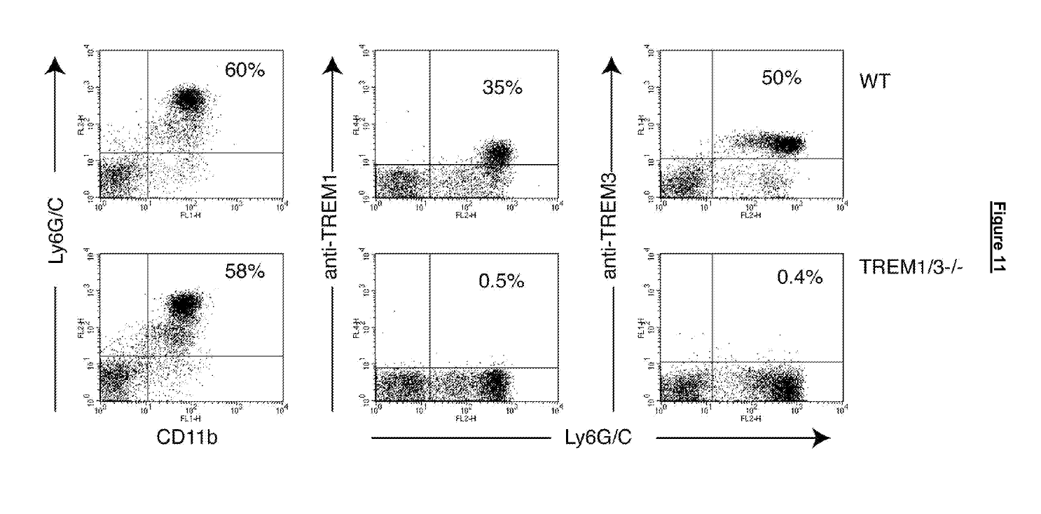

FIG. 10 shows staining results obtained with newly generated anti-TREM-3 antibodies. HEK293 cells transfected with TREM-3 (left) or untransfected HEK293 cells were stained with either mAb 87.1 or mAb 12.7 (filled histograms) or control antibody (open histograms). Both antibodies specifically recognize the TREM-3 receptor.

FIG. 11 shows flow cytometric results of bone marrow granulocytes of WT and TREM-1/-3 mice. Whole bone marrow was stained with anti-CD11b, -Ly6G-C (GR-1), -TREM-1 and -TREM-3 antibodies. Stained cells were analyzed by flow cytometry. All mice exhibit a similar population of CD11b+/Ly6G-C+ granulocytes (left column). WT granulocytes express TREM-1 and TREM-3 (middle and right columns, top panels) whereas TREM-1/3−/− do not.

FIG. 12 shows survival of TREM-1/3−/− mice. TREM-1/3+/+ and TREM-1/3−/− mice (both from parallel breedings to give homogenous 70%C57BL/6/30% 129Ola background) were subjected to a CLP sepsis challenge. Mice were subjected to a 2× #25 CLP injury and monitored for survival. TREM-1/3−/− mice were resistant to CLP as compared to wild type (WT).

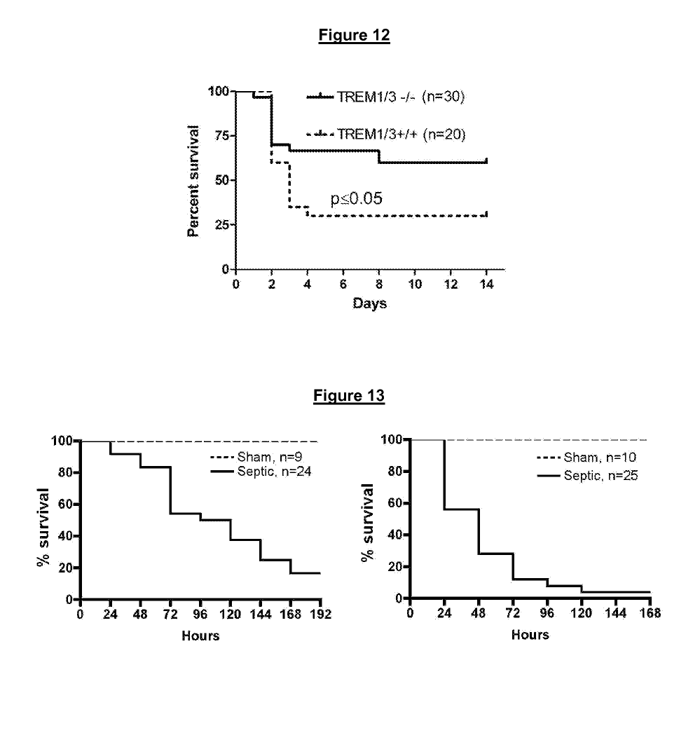

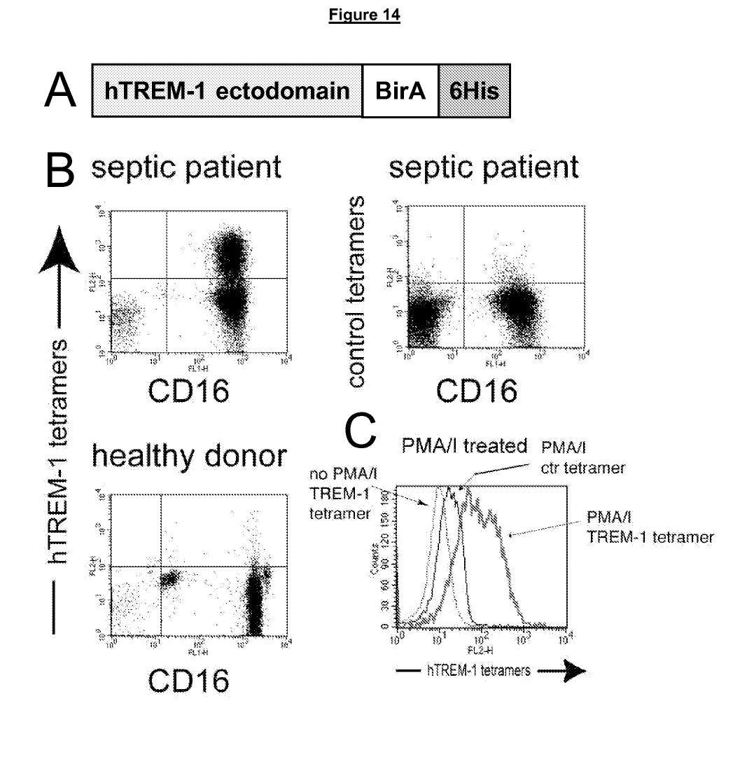

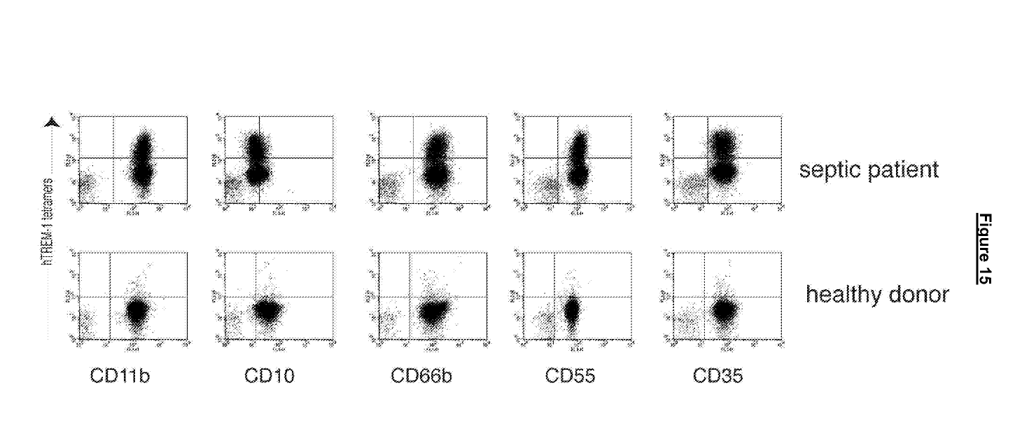

FIG. 13 shows % survival in respect of a murine model of pulmonary sepsis using a Streptococcus pneumoniae or Pseudomonas aeruginosa challenge. WT mice were subjected to pneumonia from streptococcus pneumoniae (2×10⁷ CFU of strain 99.55, left panel) or pseudomonas aeruginosa (2-4×10⁷ CFU of ATCC strain 27853, right panel) instilled by intratracheal injection. Sham mice were injected with an identical volume of sterile saline (n=9-10). Mice were observed for survival. Curves represent dosages titrated for 90% survival.

FIG. 14 shows that TREM-1 ligand is expressed on neutrophils during sepsis or in vitro activation with PMA/ionomycin.

FIG. 14(A) shows a TREM-1 tetrameric construct. The carboxy terminus of TREM-1 ectodomain is fused with a BirA tag and a 6-histidines tag. After biotinylation of the BirA sequence, TREM-1 monomers are assembled into fluorescent tetramers using PE-labeled streptavidin.

FIG. 14(B) provides a FACS analysis of neutrophils purified from blood and stained with TREM-1 tetramer and anti-CD16 antibody. TREM-1 tetramers bind a subset of CD16+ neutrophils from a septic patient but not neutrophils from a healthy donor. As proof of the specificity of TREM-1 tetramer binding, control tetramers (CD69) fail to bind human neutrophils obtained from a patient with sepsis. Note that the neutrophils of the septic patient express lower levels of CD16 in comparison to neutrophils of the healthy donor.

FIG. 14(C) shows a FACS analysis of neutrophils activated with PMA/I. TREM-1 tetramers bind neutrophils of a healthy donor after treatment with PMA/I, whereas they do not bind unstimulated neutrophils. Control tetramers do not bind neutrophils stimulated with PMA/Ionomycin.

FIG. 15 shows a FACS analysis of the hTREM-1 ligand positive subpopulation of neutrophils isolated from septic patients. These cells were positive for CD11b, CD10, CD66b, CD55 and CD35, all markers known to be expressed on circulating mature neutrophils. Neutrophils from a healthy donor also express these markers but do not bind hTREM-1 tetramer.

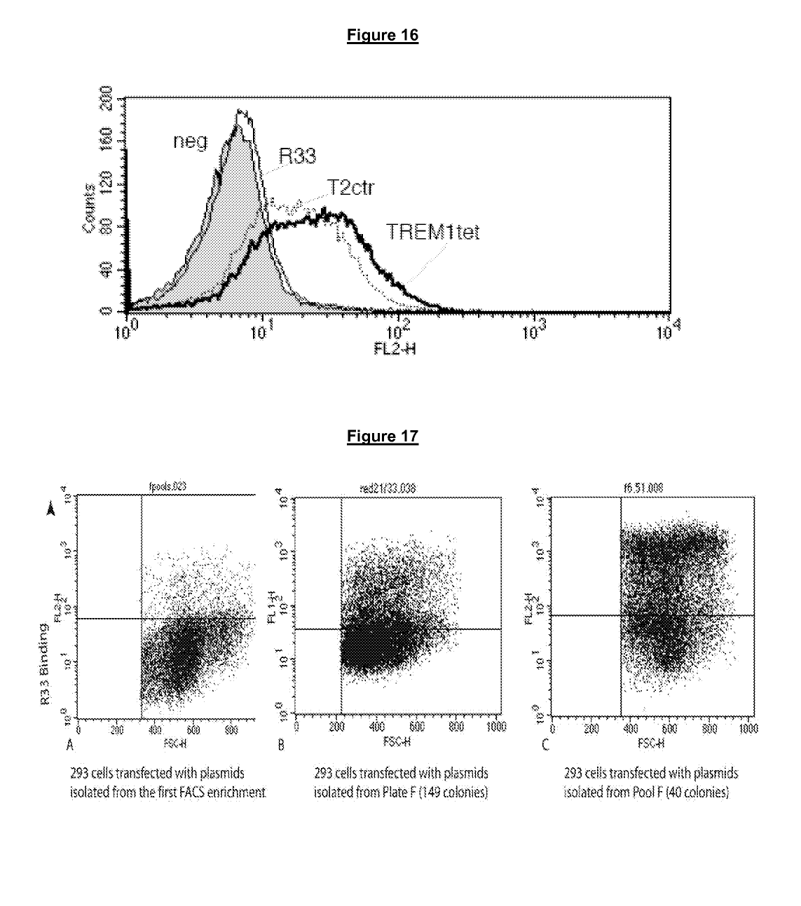

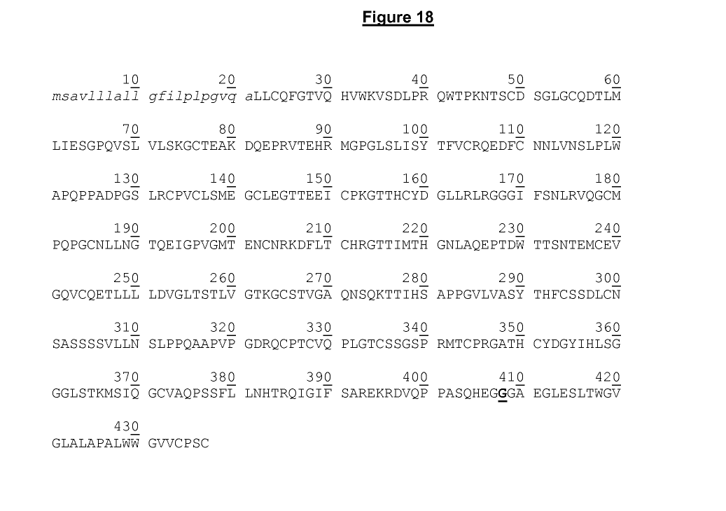

FIG. 16 shows that monoclonal antibody R33 blocks binding of TREM-1 tetramers on septic neutrophils. Neutrophils isolated from septic patients were preincubated with either R33 or an isotype matched control antibody (T2ctr). Preincubation with R33 abrogates binding of TREM-1 tetramers while the isotype matched control has no impact on tetramer binding. Thus, mAb R33 recognises the TREM-1 ligand on the cell surface.

FIG. 17 shows the results of screening a buffy coat cDNA library from sepsis patients for R33 antigen expression using the R33 monoclonal antibody. Panel A: FACS analysis of 293 cells transiently transfected with plasmids isolated from the human buffy coat cDNA library following FACS sorting of R33 positive cells. These cells are stained with R33 followed by goat anti rat Ig conjugated to PE. Panel B: Enrichment of R33 positive cells following 293 transfection with plasmids isolated from Plate F (149 colonies). Panel C: Further enrichment of R33 positive cells from Plate F.

FIG. 18 shows a human CD177 amino acid sequence. The signal peptide (amino acid 1-21) is shown in italics. A GPI-anchor amidated glycine is shown in bold and is underlined (amino acid 408).

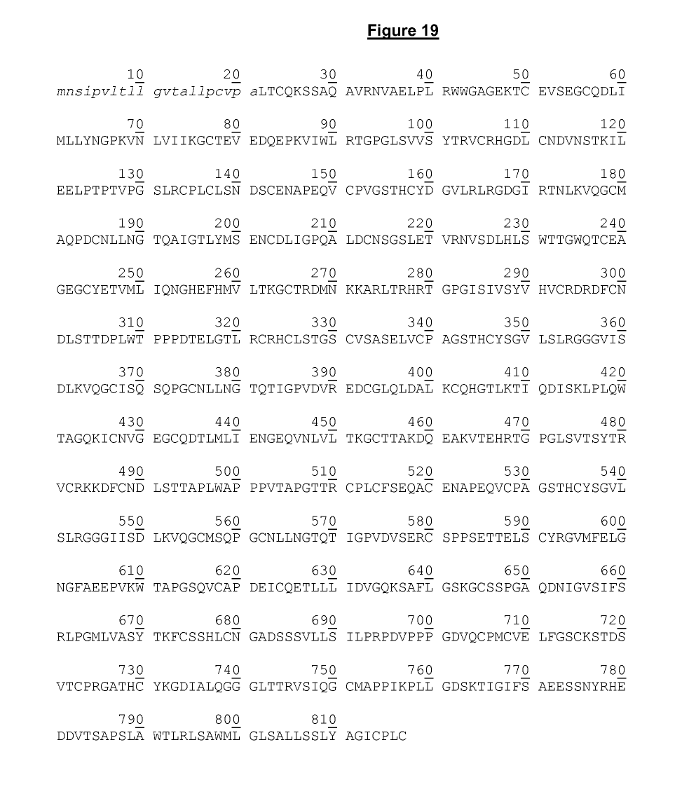

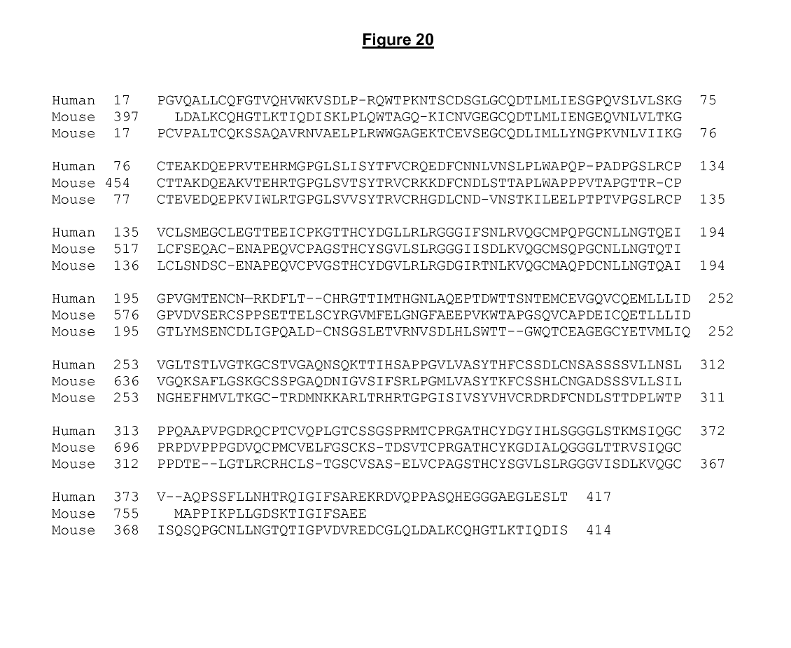

FIG. 19 shows the mouse CD177 amino acid sequence. The signal peptide (amino acid 1-21) is shown in italics. The mouse sequence is approximately twice as long as the human sequence (excluding the leader sequence). This is likely have arisen due to a gene duplication event, because two parts of the sequence have a high degree of sequence identity with each other and with the human CD177 sequence, as is best illustrated in FIG. 20.

FIG. 20 shows an alignment between the human CD177 amino acid sequence and each of two parts of the mouse sequences. It can be seen that the human sequence has significant sequence identity with each part of the mouse sequence. This may indicate a gene duplication event in the mouse.

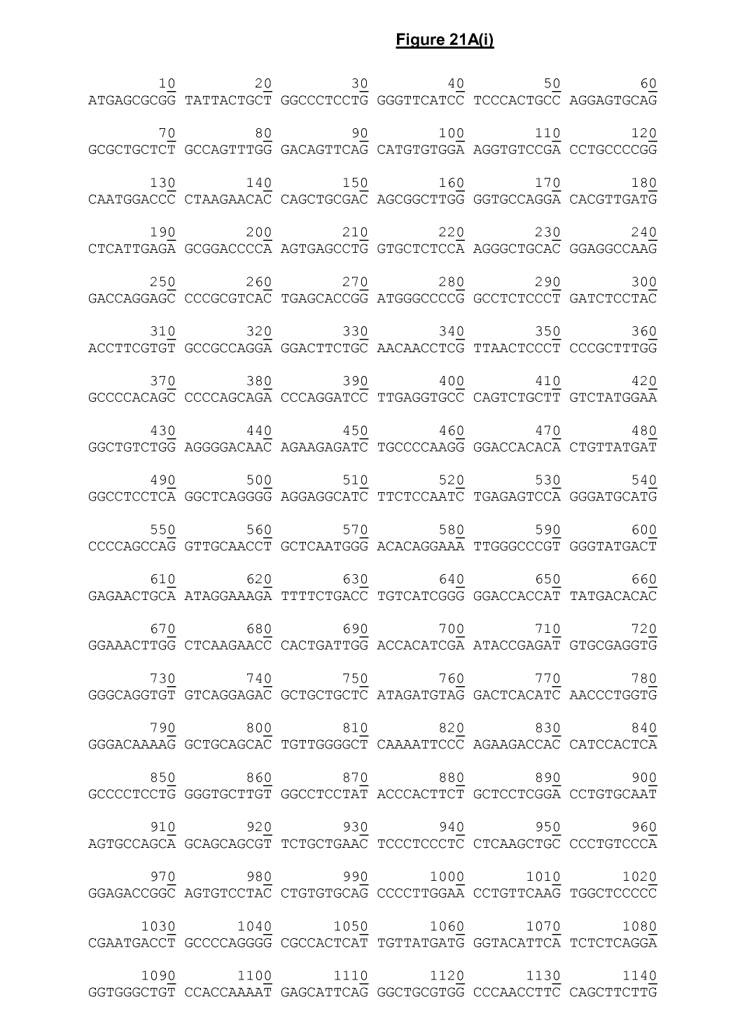

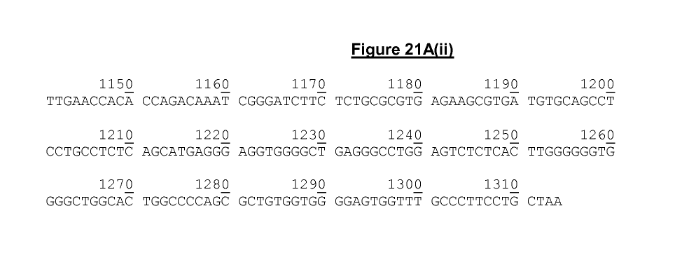

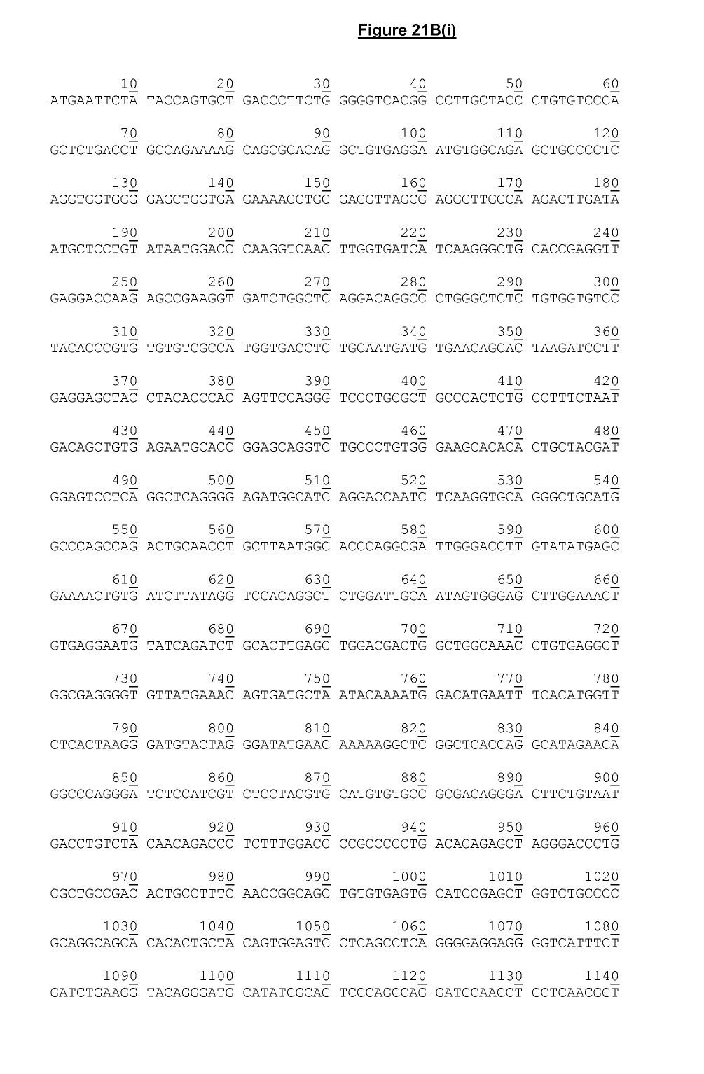

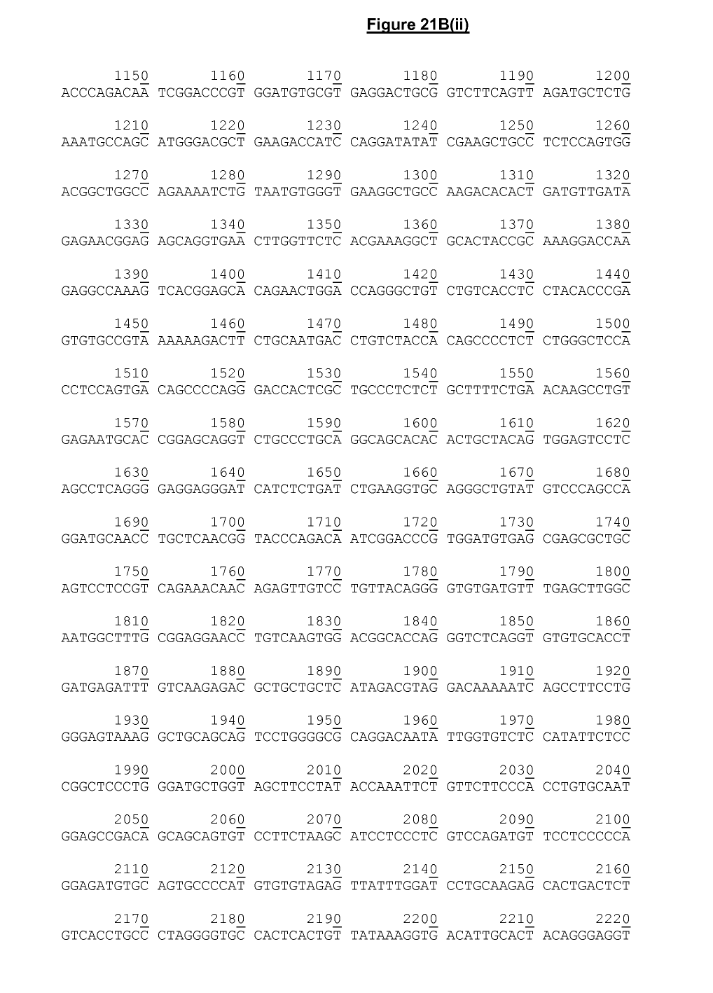

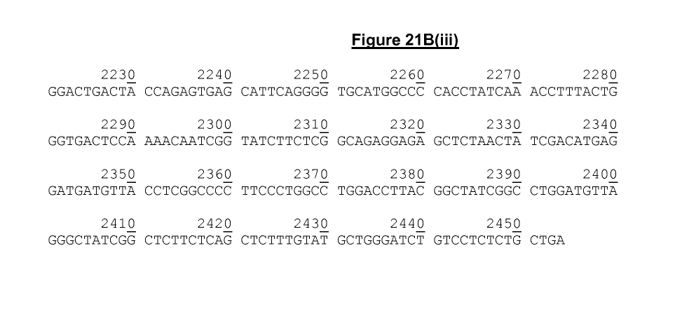

FIG. 21A shows a cDNA nucleotide sequence encoding the human CD177 amino acid sequence shown in FIG. 18.

FIG. 21B shows a cDNA nucleotide sequence encoding the mouse CD177 amino acid sequence shown in FIG. 19.

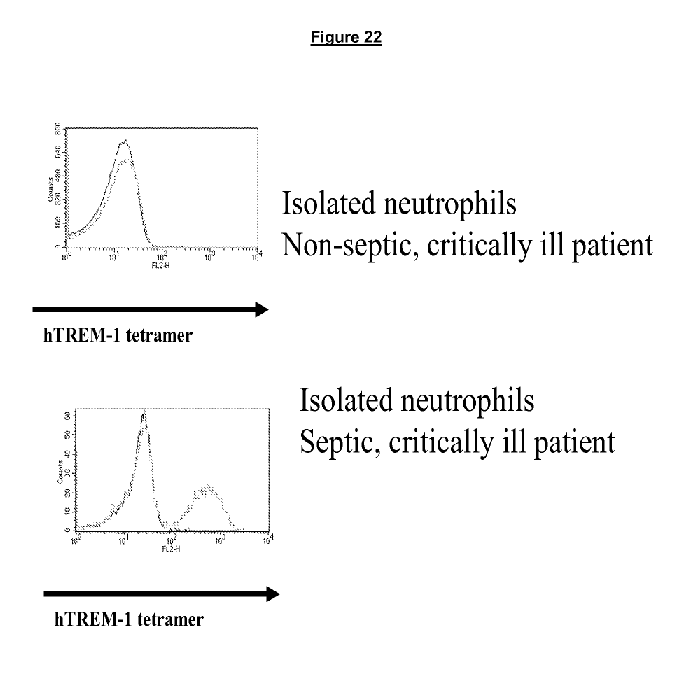

FIG. 22 shows that a TREM-1 tetramer binds to septic patient neutrophils and not to resting neutrophils.

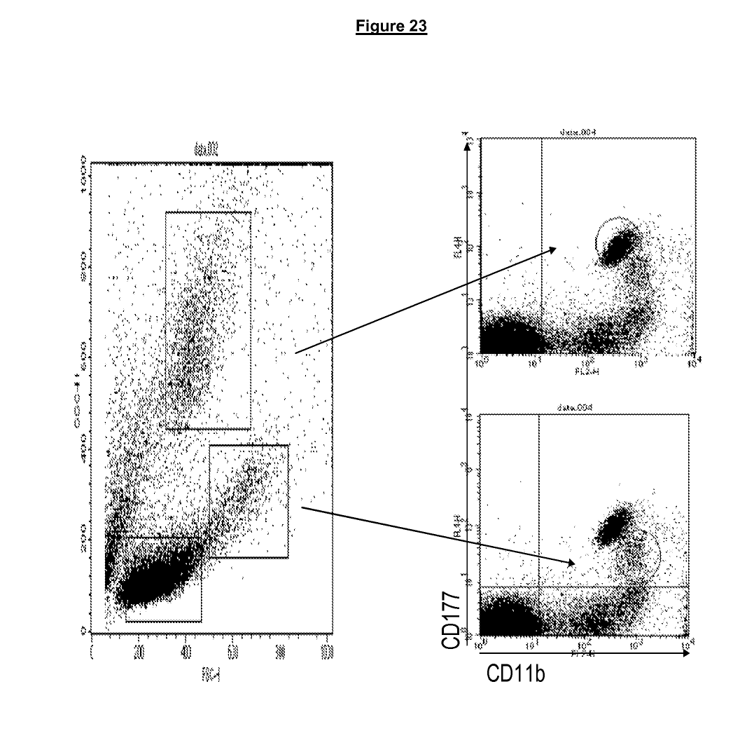

FIG. 23 shows that the anti-mCD-177 antibody Y176 binds to neutrophils and a subset of monocytes in murine peripheral blood.

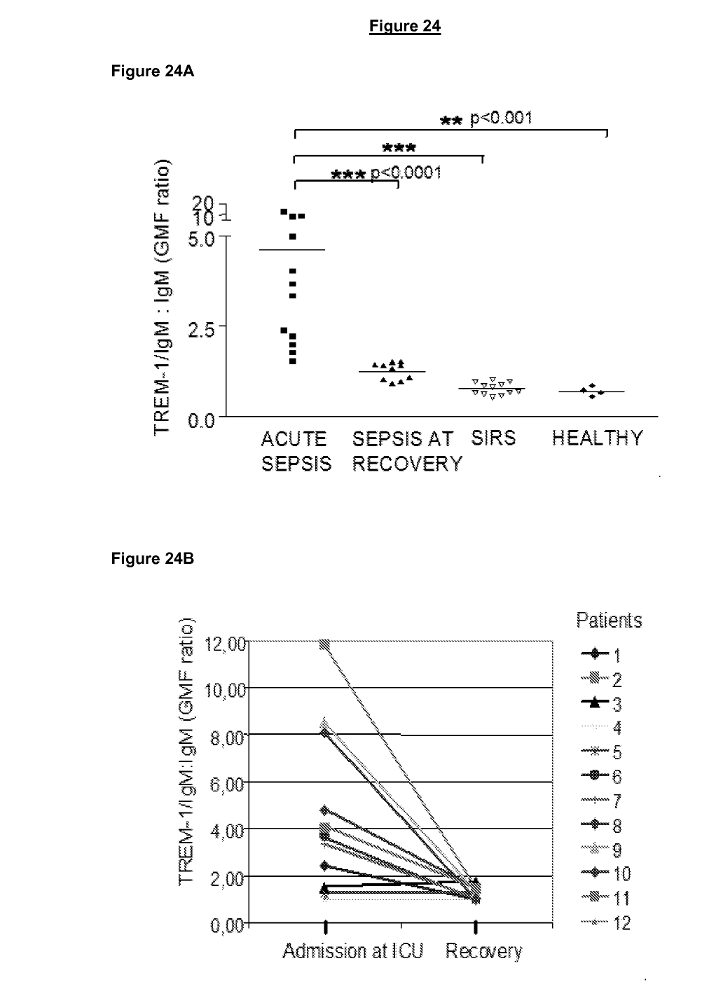

FIG. 24A shows TREM-1 ligand is specifically expressed on peripheral neutrophils of patients with sepsis. The ratio between the geometric mean fluorescence of staining with TREM-1/IgM and human IgM is reported (GMF ratio). Black squares represent patients at the time of admission into the ICU. Black triangles represent the same patients at the time of clinical recovery. White triangles represent patients with SIRS and no sign of infection. Black diamonds represent healthy individuals. Each data point represents GMF ratio of a single patient. Horizontal bars represents mean GMF values. Statistical analysis was performed by Kruskal-Wallis and Dunn's tests.

FIG. 24B shows that TREM-1 ligand expression is downregulated after recovery from sepsis. Levels of expression of TREM-1 ligand were evaluated in patients with sepsis soon after admission into the ICU and at the time of clinical recovery. Data are expressed as the ratio between the geometric mean fluorescence (GMF) of cells stained with hTREM-1/IgM versus cells stained with control hIgM.

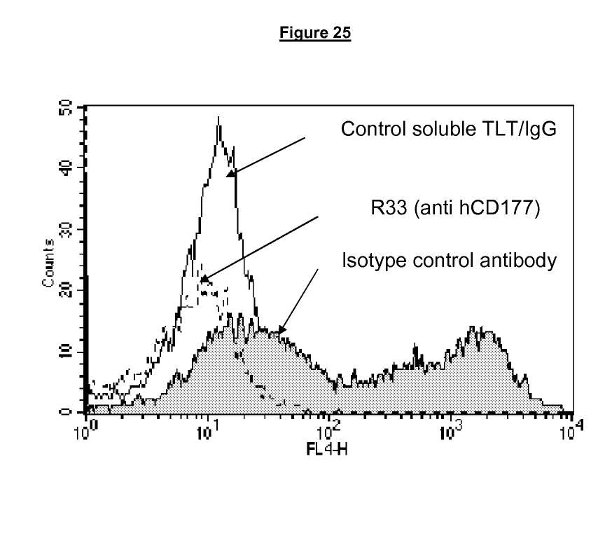

FIG. 25 shows that R33 (anti-human CD177) blocks mTREM-1 binding to hCD177 transfected HEK293 cells.

FIG. 26 shows that mouse CD177 is expressed on neutrophils and monocytes.

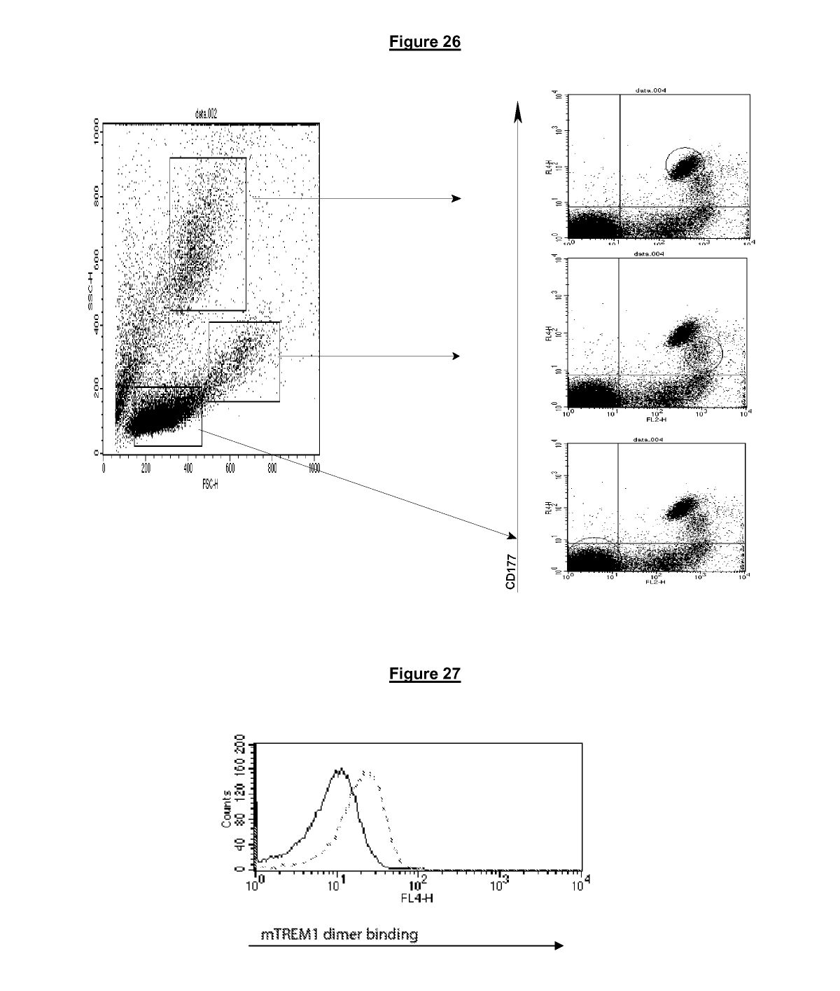

FIG. 27 provides evidence of mTREM-1 binding to mCD177 (see discussion in Example 20).

Before discussing the examples in detail, it is helpful to provide further background in respect of the function of TREM-1 and its significance. This is done below:

### BACKGROUND TO THE EXAMPLES

1. Role of TREM-1 in Sepsis

In response to tissue damage or microbial products, the innate immune system initiates an inflammatory response tasked to eradicate the invading microbes¹⁻⁴. In the context of disseminated infection or extensive tissue damage this immune response can become dysregulated, precipitating a systemic inflammatory response and a compensatory anti-inflammatory response⁵⁻⁹. The clinical consequence of this inappropriate immune activation is the sepsis syndrome, characterised by hypotension, organ failure and death^{5,7,9}. Efforts to modulate the immune system during sepsis have met with limited success, and the “magic bullet” mediator of sepsis remains unidentified^{10,11}. Recently, our group discovered the Triggering Receptor Expressed on Myeloid Cells 1 (TREM-1), a molecule expressed on neutrophils and monocytes¹². We originally observed that when TREM-1 is engaged with an agonistic antibody, proinflammatory cytokines are released¹³. In a subsequent study, we found that blockade of TREM-1 attenuates inflammation and dramatically decreases mortality in a clinically relevant experimental model of sepsis¹⁴. Additional studies found that TREM-1 is not required to initiate a response to microbial products, but instead suggest a model in which ligation of TREM-1 by its ligand causes the amplification of the immune response, synergizing with the Toll-like receptors (TLRs) and the Nod like receptors (NLR) to cause exaggerated cytokine release^{12,15,16}. These data suggest that during sepsis, modulating TREM-1 could attenuate inflammation without causing immunoparalysis or inhibiting antimicrobial function and mandate a systematic examination of the role of TREM-1 in sepsis.

2. The TREM Family.

TREM-1 is the founding member of a family of receptors expressed in granulocytes (neutrophils), monocyte/macrophages, dendritic cells (DC), osteoclasts and microglia called triggering receptors expressed on myeloid cells (TREM)^{12,17-19}. TREMs are transmembrane glycoproteins of the immunoglobulin (Ig) superfamily encoded by a gene cluster that maps to human chromosome 6p21 and mouse chromosome 17C3 in linkage with the MHC (FIG. 1)^{12,19,20}. The TREM gene cluster encodes both activating and inhibitory receptors.

Activating TREM-1, TREM-2 and TREM-3 receptors contain a single extracellular Ig-like domain of the V-type, a transmembrane region with a charged residue (lysine) and a short cytoplasmic tail (FIG. 1). TREM-3 exists only as a pseudogene in humans (FIG. 1). TREM-1, TREM-2 and TREM-3 associate with the protein adaptor DAP12 for cell surface expression, signalling and function (FIG. 2). The cytoplasmic domain of DAP12 contains an immunoreceptor tyrosine-based activation motif (ITAM), which functions as docking site for protein tyrosine kinases Syk and ZAP70²¹⁻²³. These promote recruitment and tyrosine phosphorylation of multiple adaptors and downstream signalling mediators, leading to intracellular Ca²⁺ mobilization, rearrangement of the actin cytoskeleton, activation of transcription factors, and, ultimately, to cell activation (FIG. 2).

The TREM cluster includes at least two other genes encoding the TREM-related proteins, called TLT-1 and TLT-2. TLT-1 is expressed in platelets, contains an immunoreceptor tyrosine-based inhibitory motif (ITIM) in its cytoplasmic tail and recruits protein tyrosine phosphatases²⁴⁻²⁵ (FIG. 1). TLT-2 is expressed in B cells and macrophages, contains a proline-rich region in its cytoplasmic tail and its function is unknown²⁶ (FIG. 1). Additional TREM-like genes and pseudogenes have been predicted by computational analysis of the TREM genomic region (FIG. 1). TREMs share limited homology with other Ig gene superfamily members. The closest TREM relative is NKp44, an activating NK cell receptor encoded by a gene closely linked to the TREM genes²⁷. More distant relatives of TREMs include the CMRF-35 family members²⁸⁻³¹. The receptor for polymeric Ig (plgR)³² also shares homology with the extracellular region of the TREM family. However, none of the TREM receptors, CMRF35 or NKp44 bind Ig. In fact, the ligands for all these receptors are as yet unknown.

Among TREMs, TREM-1 and TREM-2 are the most extensively characterised. TREM-2 is primarily expressed in preosteoclasts and microglia¹². A genetic defect in TREM-2 results in a human disease, Nasu-Hakola disease (NHD), characterised by severe bone abnormalities and brain demyelination³³. TREM-2 is also expressed in bone marrow derived macrophages, thioglycollate-elicited macrophages and alternatively activated macrophages ^{34,35} and modulates their cytokine responses to microbial products^{35,36}. TREM-1 is expressed in granulocytes (neutrophils) and monocytes/macrophages. Preliminary studies in human suggest that TREM-1 activates these cells in vitro and contributes to systemic inflammatory responses and sepsis during microbial infections in vivo^{13,14}.

3. TREM-1 Amplifies Inflammation and Contributes to Pathogenesis of Sepsis.

Human TREM-1 is expressed on blood neutrophils and a subset of monocytes. In normal tissues, TREM-1 is selectively expressed on alveolar macrophages. These are long-lived effector cells in the lung, specialized in recognition and clearance of pathogens, phagocytosis of apoptotic or damaged cells and removal of macromolecules. Furthermore, TREM-1 is expressed at high levels in neutrophilic infiltrates and epithelioid cells in human skin and lymph nodes infected by Gram positive and Gram negative bacteria as well as fungi^{14,37}. The tissue distribution of TREM-1 expression has suggested a role in inflammation. Consistent with this, we have shown that engagement of TREM-1 on human granulocytes and monocytes with agonist mAbs stimulates production of pro-inflammatory chemokines and cytokines. IL-8, a potent chemoattractant for neutrophils, is strongly induced by engagement of TREM-1. Monocyte chemoattractant protein-1 (MCP-1), MCP-3 and macrophage inflammatory protein 1α (MIP-1α) are also induced. TREM-1 triggering induces granulocyte release of myeloperoxidase but not phagocytosis. Moreover, TREM-1 and TLRs cooperate with each other in inducing inflammation. Monocyte secretion of TNF-α and IL-1α in response to LPS is markedly upregulated when TREM-1 mAbs are used as a co-stimulus, demonstrating the ability of TREM-1 to amplify inflammatory responses initiated by TLR^{13,15}. In addition, LPS and other TLR ligands upregulate TREM-1 expression, potentiating its pro-inflammatory function^{13,15}.

To address the role of TREM-1 as an amplifier of inflammation in vivo, we generated a recombinant mouse soluble TREM-1 fused with the Fc part of human IgG1 (mTREM-1-Fc). This TREM-1-Fc should compete with the endogenous TREM-1 for binding TREM-1 ligands, neutralizing the biological activities of endogenous TREM-1. In an animal model of LPS-induced endotoxemia, blocking TREM-1 signalling with mTREM-1-Fc reduced hyper-responsiveness and death¹⁴. In models of septic shock, including intraperitoneal injection of live E. coli and caecal ligation and puncture (CLP), blocking TREM-1 also protected mice against shock and death¹⁴.

Further corroborating a proinflammatory role of TREM-1, transgenic mice overexpressing the TREM-1 signalling adaptor, DAP12, developed high numbers of blood neutrophils as well as massive macrophage infiltration in the lung and are highly susceptible to LPS-induced shock³⁸. This phenotype may be explained in part by constitutive activation of the TREM-1/DAP12-dependent pathway. Moreover overexpression of DAP12 increased hepatic granulomatous inflammation elicited by zymosan A, while blockade of TREM-1 reduced granuloma formation³⁹. Together, these results highlight the crucial role of TREM-1 in the amplification of inflammatory responses by granulocytes and macrophages, particularly in response to microbial components, and implicate TREM-1 as a potential target for therapeutic intervention in human diseases caused by excessive inflammatory responses to infections, such as septic shock.

4. Soluble TREM-1 Mimetics Modulate Inflammation.

A soluble form of TREM-1 (sTREM-1) has been identified in the serum of patients with sepsis⁴⁰ as well as in the serum of animals involved in an experimental model of septic shock⁴¹. Moreover, sTREM-1 was detected in the bronchioalveolar lavage (BAL) of patients with pneumonia⁴². There are two possible origins for sTREM-1. One possibility is that sTREM-1 is generated by proteolytic cleavage or membrane shedding of surface expressed TREM-1. Alternatively, sTREM-1 may be generated by de-novo translation of an TREM-1 mRNA splice variant which codes for a secreted form of TREM-1. In support of this latter hypothesis, an alternative TREM-1 transcript which lacks exon 3 that encodes the transmembrane region, has been reported^{43,44}. The physiological role of sTREM-1 is not fully understood. This molecule may scavenge the TREM-1 ligand that is not immediately bound to the surface displayed TREM-1, thereby blunting immune responses and providing local control in the setting of inflammation⁴⁰. Indeed, controlled release of soluble forms of multiple receptors critical to immunologic signalling and the inflammatory response has been described. These include a soluble form of the IL-1 receptor (IL-1 decoy RII)^{45,46}, TNF-α receptor⁴⁷⁻⁵⁰, and L-Selectin receptor⁵¹. Consistent with a modulatory function of sTREM-1, a synthetic peptide mimicking a short highly conserved domain of sTREM-1 (LP17, TDSRCVIGLYHPPLQVY) attenuated cytokine production by human monocytes in vitro and protected septic animals from hyper-responsiveness and death in vivo⁴¹. This peptide was efficient not only in preventing sepsis but also in treating sepsis once the deleterious effects of proinflammatory cytokines is initiated. These data suggest that in vivo modulation of TREM-1 by a sTREM-1 peptide could be a suitable therapeutic tool for the treatment of sepsis.

5. TREM-1/DAP12 Signalling Promotes Granulocytes and Macrophage-Inflammatory Responses.

How does TREM-1 elicit inflammatory responses? Human TREM-1 transmembrane region contains a charged residue (lysine) that allows association with the adapter DAP12⁵². DAP12 contains cytoplasmic immunoreceptor tyrosine-based activation motifs (ITAM) (FIG. 2). Engagement of a DAP12-associated receptor induces tyrosine phosphorylation of the ITAM by Src kinases. The phosphorylated ITAM recruits the protein tyrosine kinases Syk and ZAP70, triggering phosphorylation of multiple adaptors such as LAT, NTAL, Slp76 in complex with Vav and Grb-2 in complex with Sos. DAP12 also induces activation of phosphatidylinositol 3-kinase (PI3-K), phospholipase Cγ1/2 (PLCγ1/2), TEC kinases, c-Cbl, and other downstream signalling mediators²¹⁻²³. These cytoplasmic mediators trigger intracellular Ca²⁺ mobilization, rearrangement of the actin cytoskeleton, activation of extracellular-signal-regulated kinases (ERK) 1/2 and transcription factors, ultimately activating cell effector functions (FIG. 2).

Studies measuring the response of cells to the ligation of DAP12-associated receptors suggest that DAP12 signalling alone only triggers a limited inflammatory response. However, DAP12 strongly synergizes with other signalling pathways activating inflammation, particularly those triggered by TLRs. Typically, TLRs signal through the adapters MyD88 and TRIF (FIG. 3). MyD88 recruits IRAK4, IRAK1 and TRAF6, initiating a signalling cascade ultimately leading to activation of NF-kB⁴. TRIF recruits TBK-1 and IKKε, which mediate phosphorylation of IRF-3 and transcriptional activation of IFN-β⁴. Moreover, TLRs signal through Src tyrosine kinases via MyD88-dependent and -independent pathways⁵³. DAP12-mediated signalling clearly potentiates TLR-mediated inflammatory responses in vitro and in vivo. However, the mechanisms for this synergy are poorly understood. It has been shown that sustained ERK activation is essential for activation of the transcription complex AP-1, particularly c-Fos^{54,55}. Moreover, DAP12-signalling induces sustained intracellular calcium mobilization, which activates Ca²⁺/calmodulin-dependent phosphatase calcineurin^{56,57}. Calcineurin dephosphorylates nuclear factor of activated T cell (NFAT) transcription factors, leading to their nuclear translocation^{56,57}. Thus, DAP12-mediated AP-1 and NFAT activation may synergize with NF-kB activation induced by TLR, resulting in enhanced transcriptional activation of genes encoding inflammatory mediators (FIG. 3). A similar model was recently demonstrated in osteoclasts, where DAP12 and other ITAM-containing adaptors generate Ca²⁺ signals that allow Receptor Activator of NF-kB (RANK) to induce of NFATc1 (NFAT2), a key transcription factor for osteoclastogenesis⁵⁸.

β1 and β2 integrins expressed on the cell surface of granulocytes and macrophages also contribute to inflammation by mediating cell-cell interactions and adhesion to extracellular matrix proteins^{59,60}. The adhesive function of integrins depends on intracellular signals generated by chemokine receptors that modify conformation and surface distribution of integrins⁶¹. It is possible that DAP12 also generates intracellular signals, such as Vav phosphorylation, that contribute to integrin activation (FIG. 3). Proinflammatory responses of granulocytes and macrophages are elicited through additional receptors, such as those for IgG Fc, formyl-peptides, the inflammatory cytokines IL-1 and TNF, CD40L and other TNF-superfamily members. The impact of DAP12-signalling on these diverse signalling pathways has not been investigated.

6. Constructing a Mouse Model of TREM-1: Significant Differences Between Human and Mouse Genes.

DAP12 is a transmembrane signalling adaptor associated with a family of activating immunoreceptors including not only the TREMs, but also SIRP-β1, CD200R, MDL-1, KIRs, Ly49s, NKG2C/E, and others⁶². These receptors are expressed on the surface of granulocytes (neutrophils), macrophages, DC, osteoclasts, microglia and NK cells. To validate the function of TREM-1 in sepsis in vivo it is necessary to develop a TREM-1 knockout mouse. However, in the case of the mouse, the TREM-1 gene is adjacent to a highly homologous gene, TREM-3, likely the result of a gene duplication event. TREM-3, is separated from TREM-1 by only 4 kb, is expressed in mouse macrophages and is strongly upregulated in response to LPS¹⁹. Like TREM-1, TREM-3 promotes cell activation through DAP12¹⁹. It is reasonable to assume that, given their sequence homology and structural similarities, these two gene products have similar or overlapping functions in the mouse and may recognize the same ligand or closely related ligands. In contrast, in human, TREM-3 is a pseudogene, it is not expressed at the protein level and therefore there is no potential overlap between TREM-1 and TREM-3. Thus, to model the effect of blocking TREM-1 in humans, we have generated a TREM-1/TREM-3 double knockout (TREM-1/3−/−) mouse. Our results show that TREM-1/3−/− mice are more resistant to CLP than WT mice, indicating that the TREM-1/3-DAP12 signalling complex exacerbates inflammation in the context of authentic sepsis.

7. Activating vs Inhibitory DAP12 Signalling: The Opposing Roles of TREM-1 and TREM-2.

In contrast to the demonstrated role of DAP12 in activating cells, Hamerman et al. have recently reported that in bone-marrow derived macrophages, DAP12 can inhibit TLR-mediated cytokine production and that DAP12−/− mice are more sensitive to LPS than WT mice, when LPS is co-administered with the TNF-α sensitizing reagent D-galactosamine⁶³. Thus, these results paradoxically suggest an inhibitory role for DAP12 in regulating the TLR response to LPS. In our data and in reference⁶⁴, we demonstrate that in physiological models of bacterial-induced inflammation, DAP12−/− mice are more resistant to CLP than WT mice.

Moreover, DAP12−/− peritoneal exudate cells (PEC) recovered from septic mice produce less cytokines than wild type PECs. These results confirm a pro-inflammatory function of DAP12, at least in the presence of bacterial infections. We further find that TREM-1/3−/− mice are resistant to CLP as compared to WT, suggesting that TREM-1/3-DAP12 significantly contributes to the inflammatory response in-vivo.

Recently, new studies by Hamerman et al.³⁶ and our group³⁵ identified TREM-2 (as opposed to TREM-1/3) as the receptor mediating the inhibitory effects of DAP12 during the in-vitro stimulation of bone-marrow derived macrophages with low concentrations of LPS, and possibly also in D-galactosamine potentiated endotoxemia. Using an in vitro transfection system, Hamerman et al. found that small interfering RNA (siRNA)-mediated inhibition of TREM-2 expression increased the response of macrophages to TLR agonists, and that this function of TREM-2 required an intact ITAM³⁶. Studies by our group drew identical conclusions from the analysis of wild type (WT) and TREM2−/− mice, observing that macrophages derived from TREM2−/− mice have an increased TLR-mediated cytokine response compared with those from WT mice, and that this effect completely accounted for the increased cytokine production previously observed in DAP12−/− mice³⁵. Altogether, these data conclusively identify TREM-2 as mediating the inhibitory effect of DAP12 on macrophages.

These data resolve the apparent conflict in the data on the role of DAP12 in simple endotoxemia vs. D-galactosamine-potentiated endotoxemia. We hypothesize that the activating vs. inhibitory effects of DAP12 reflects the involvement of TREM-1 vs TREM-2 and differences in the affinity or avidity of these receptors for their ligand(s). In the case of simple endotoxemia and CLP, high doses of LPS (in a dose range of 5-10 mg/kg) and authentic bacterial infection induce expression of a high-affinity TREM-1 ligand and full TREM-1/DAP12 phosphorylation, leading to activation of granulocytes and monocytes and an increase in the magnitude of the inflammatory response (FIG. 3). In the case of D-galactosamine-potentiated endotoxemia, low doses of LPS (20-100 ng, 1000× less than in simple endotoxemia) induce low avidity TREM-2 ligand(s) that trigger inhibitory TREM-2 signalling, attenuating the inflammatory response and improving survival (FIG. 3). In support of this model, recent data demonstrate that both TREM-2³⁵ and a putative low affinity TREM-2 ligand³⁶ are expressed on the surface of macrophages, suggesting that TREM-2 could provide a tonic inhibitory signal to macrophages. Inhibitory signalling may be mediated by incomplete DAP12 phosphorylation and consequential recruitment of protein tyrosine phosphatase SHP-1, a major cytosolic mediator of inhibition^{65,66}.

### EXAMPLE 1

### DAP12 Signalling Contributes to Inflammation and Mortality from Endotoxemia

It has previously been shown that antibody ligation of the DAP12-associated receptor TREM-1 on granulocytes and monocytes amplified LPS-induced release of inflammatory cytokines TNF-α and IL-8^{13,15}. In-vivo, blockade of the DAP12-associated receptor TREM-1 was associated with reduced inflammation and increased survival from endotoxemia or septic peritonitis¹⁴. These observations suggest a role for TREM-1 and its associated adaptor DAP12 in the amplification of the inflammatory response induced by pathogens and their components. To corroborate this hypothesis, we sought to understand the function of DAP12 in physiologically relevant models of inflammation. To this end we measured the contribution of DAP12 to septic shock induced by endotoxemia and CLP.

To determine if DAP12 contributed to the in-vivo response to endotoxin, we subjected WT and DAP12−/− mice to intraperitoneal injection of LPS and monitored them for survival. DAP12−/− mice tolerated doses of 5 mg/kg and 6.25 mg/kg of endotoxin, which resulted in 60-100% mortality in WT mice (FIG. 4A). However, DAP12−/− mice were not completely refractory to endotoxin, as they succumbed to a dose of 10 mg/kg (FIG. 4A). Thus, DAP12 signalling contributes to endotoxemia, although it is not required for the response to LPS.

Endotoxin causes shock by inducing macrophage production of TNF-α and other proinflammatory cytokines⁶⁷. To determine if DAP12 signalling exacerbated endotoxemia by increasing cytokine production, we measured cytokine levels in mice treated with 5 mg/kg endotoxin. It has been shown that inflammatory cytokine levels peak in 1-3 hours of endotoxemia⁶⁸. We found that 2 hours after injection of LPS, both WT and DAP12−/− mice had elevated circulating levels of TNF-α, IL-6, MCP-1 and IL-10. When compared to DAP12−/− mice, WT animals had significantly higher levels of TNF-α and IL-10. By 4 hours, TNF-α and IL-10 were reduced in WT mice and the levels were equal to the DAP12−/− animals (FIG. 4B).

We conclude that DAP12 signalling can augment inflammatory cytokine production acutely.

### EXAMPLE 2

### Mortality and Inflammation from Septic Peritonitis is Augmented by DAP12

Whereas mortality from endotoxin is mediated by inflammation and overproduction of cytokines, surviving authentic sepsis requires attenuating the inflammatory response, as well as achieving bacterial control⁶⁹. To determine the role of DAP12 in survival of sepsis, we subjected WT and DAP12−/− mice to CLP, a clinically relevant model of bacterial peritonitis. We found that DAP12−/− mice were highly resistant to CLP (WT, n=20, no survival; DAP12−/−, n=19, 60% survival; FIG. 5A).

Sepsis is associated with high circulating cytokine levels that contribute to shock⁶⁹. To determine if DAP12 contributed to cytokine production during sepsis, we measured cytokine levels in the serum of WT and DAP12−/− mice 6 and 24 hours after CLP. At 6 hours, WT and DAP12−/− mice had measurable serum levels of IL-6, MCP-1 and TNF-α, but there was no difference between the two groups. Between 6 and 24 hours, WT serum cytokine levels increased dramatically such that by 24 hours after the onset of sepsis the WT mice had significantly higher levels of IL-6, MCP-1, TNF-α and IL-10 than did DAP12−/− mice (FIG. 5B). These data demonstrate that DAP12 signalling contributes to cytokine production during sepsis.

To determine if there were other DAP12-regulated factors mediating the increased sepsis mortality of WT mice as compared DAP12−/− animals, we compared plasma proteins from these mice using 2-dimensional difference gel electrophoresis (2D DIGE)⁷⁰. Plasma was isolated from WT and DAP12−/− mice 24 hours after CLP, and plasma proteins were resolved by isoelectric point and size. Relative abundance of the individual gel features was compared between the two genotypes, and features that were significantly different in 4 independent experiments were isolated and identified by mass spectrometry. We identified 7 differentially regulated proteins in 13 gel features (some proteins are represented by multiple gel features). Data are expressed as the fold change in average florescence of DAP12−/− vs. WT (Table I).

The proteins identified in this unbiased approach were previously described as acute phase reactants. Positive acute phase proteins (proteins known to increase in response to inflammation, i.e. apolipoprotein A-IV⁷¹, hemopexin⁷² and complement component 3⁷²) accounted for 3/7 identified proteins. Negative acute phase proteins (those known to decrease with inflammation) accounted for 4/7 proteins (major urinary protein⁷³, antithrombin III⁷⁴, gelsolin⁷⁵ and MHC Q10⁷⁶). For every individual protein, the acute phase response was attenuated in DAP12−/− mice. This was manifest as lower levels of positive acute phase proteins and higher levels of negative acute phase proteins.

Taken together, these data show reduced plasma cytokine levels and a reduced acute phase response in DAP12−/− mice, demonstrating a role for DAP12 in triggering inflammation.

### EXAMPLE 3

### DAP12 is not Required for Recruitment of Cells or Bacterial Killing

We hypothesized that the absence of DAP12 could also result in a decreased cellular response to peritonitis. To address this question we measured the number and type of cells recruited to the peritoneum during sepsis. We found that equal numbers of cells in the peritoneum of WT and DAP12−/− mice 24 hours after the onset of sepsis (FIG. 6A). By analyzing surface markers on these cells, we found that in both WT and DAP12−/− mice, 50-60% of the cells were macrophages (defined as CD11b⁺ GR1^{lo}) and 30-40% were granulocytes (defined as CD11b⁺GR1^{hi}) (FIG. 6B). The absence of DAP12 does not appear to alter the recruitment of cells to the peritoneum during sepsis. We also asked if there was a deficit in bacterial control in the absence of DAP12-signalling. To determine if DAP12 mediates bacterial control, we measured bacterial load in the peritoneum at 24 hours. We found no significant difference in peritoneal infection between WT and DAP12−/− mice (FIG. 6C), demonstrating that DAP12 is not required to control the peritoneal infection during sepsis.

TABLE I
Differential Plasma Proteins Induced by Sepsis in
DAP12−/− vs. WT mice.

| | Fold Change | p-value |
|---|---|---|
| Positive Acute Phase | | |
| Reactants | | |
| Apolipoprotein A-IV | −1.56 | 0.05 |
| Hemopexin | −3.68 | 0.024 |
| Complement component 3 | −3.17 | 0.0005 |
| Negative Acute Phase | | |
| Reactants | | |
| Antithrombin | 1.69 | 0.0047 |
| Gelsolin | 1.72 | 0.0018 |
| Major urinary protein | 3.49 | 0.006 |
| MHC Q10 alpha chain | 2.13 | 0.04 |

Proteomic analysis of plasma from WT and DAP12−/− mice 24 hours after CLP identified 7 differentially regulated proteins. Identified proteins were previously described as acute phase reactants demonstrating a role for DAP12 in inducing the acute phase response during sepsis. Mean fluorescent intensity was

### EXAMPLE 4

### DAP12-Signalling Augments In-Vitro Cytokine Production and ERK Signalling of Peritoneal Exudates Cells Obtained from Septic Mice

Our results indicate that in-vivo DAP12-signalling augments cytokine production. To investigate the cellular basis of these observations, we measured cytokine production by peritoneal exudate cells induced by sepsis. Peritoneal cells were isolated by peritoneal lavage and tested for their ability to produce cytokines ex-vivo either without or with LPS stimulation. We found that cells isolated from the peritoneum of both WT and DAP12−/− after CLP produced cytokines in the absence of any additional stimulation (FIG. 7A). However, cells isolated from WT mice produced more MCP-1, TNF-α and IL-10 as compared to cells isolated from DAP12−/− mice. Although the cells are cultured in the presence of antibiotics, we cannot determine if this cytokine secretion reflects stimulation by bacterial products carried over from the frankly septic peritoneal lavage fluid or if this cytokine production reflects activation induced by previous stimulation in-vivo. To normalize the stimulation via TLRs and to exclude the possibility of differential carryover of microbial products, we treated the cells with 1 μg/mI LPS, which resulted in maximal stimulation and increased cytokine production by WT and DAP12−/− cells. Under these conditions, we found that WT cells were significantly more efficient at cytokine production than DAP12−/− cells (FIG. 7A). Remarkably, when we compared ex-vivo cytokine production by thioglycollate-elicited peritoneal exudate cells from WT and DAP12−/− mice, we found no statistically significant differences in IL-10, IL-6, MCP-1 or TNF-α (FIG. 7B), although there was a trend toward an increase in IL-10 production by DAP12−/− cells. We conclude that DAP12-signalling augments cytokine production only in PEC stimulated in-vivo by septic peritonitis.

We further investigated the signalling pathways underlying the DAP12-mediated increase of cytokine production in septic peritonitis-induced PEC. To this end, PEC were harvested 24 hours after CLP and stimulated with LPS for various times. Cell lysates were resolved by SDS-PAGE and immunoblotted for phospho-ERK1/2 and then reblotted for total ERK2 (FIG. 8). WT mice showed increased ERK phosphorylation after 30 and 60 minutes of stimulation. These data demonstrate that DAP12 signalling augments LPS-mediated ERK activation.

These studies unequivocally demonstrate that DAP12 contributes to death from septic peritonitis by increasing inflammation. In the models of diffuse peritonitis and endotoxemia, DAP12 is not required to recruit cells to the peritoneum or to mediate an antimicrobial response. We found that DAP12 signalling exacerbates the inflammatory response by amplifying inflammatory cytokine production.

### EXAMPLE 5

### TREM-1/TREM-3 Double-Deficient (TREM1/3−/−) Mice are less Susceptible to CLP than WT Mice

Our data (FIG. 4-7) clearly establish a role of DAP12 signalling in promoting inflammation. However, absence of DAP12 in mice may impact the signalling of multiple cell surface receptors expressed on inflammatory cells. These include not only TREM-1, but also SIRPβ1^{77,78}, CD200R⁷⁹⁻⁸², IREM2⁸³, MDL-1⁸⁴, and others⁶². Thus, DAP12−/− mice cannot be used to pinpoint the specific functions of TREM-1 in vivo. To address the function of TREM-1 in vivo is essential to generate a knockout model.

In the mouse, the TREM-1 gene is adjacent to a very similar gene, TREM-3¹⁹, which is likely to encode a protein which may have overlapping function with TREM-1 and may recognize the same ligand or closely related ligands. In contrast, in human, TREM-3 is a pseudogene. Thus, to best model the human TREM system in vivo, we have generated TREM-1-TREM-3 double knockout mice (TREM-1-3−/−).

The TREM-1-3 targeting construct was designed to delete exons 3 and 4 encoding the transmembrane and cytoplasmic domains that are required for association of TREM-1 with DAP12, and exon 1 of TREM-3, encoding the leader sequence of TREM-3 (FIG. 9). We electroporated the construct into E14.1 ES cells, injected correctly targeted clones into C57BL/6 blastocysts and obtained chimeras that were bred to transgenic mice expressing Cre under the CMV promoter⁸⁵. Chimeras transmitted the TREM-1-3 mutation with the neomycin resistance gene deleted. By intercrossing TREM-1/3−/+ mice we produced mice homozygous for the deletion.

To demonstrate lack of TREM-1/3 expression in our mice, we prepared blood and bone marrow granulocytes from TREM-1/3−/− mice and WT littermates and stained them with an anti-mTREM-1 mAb (50D1)¹⁴) and an anti-mTREM-3 mAb that we recently generated (FIG. 10). Flow cytometric analysis demonstrated that while WT granulocytes express high levels of TREM-1 and TREM-3 both TREM-1 and TREM-3 are completely absent on TREM-1/3−/− granulocytes (FIG. 11). To address this possibility, theTREM-1/3−/− mice were backcrossed onto the C57B1/6 background and then intercrossed when >70% of the genome was derived from the C57BL6 strain (as measured by SSLP typing). Consideration of the genetic background of the mice is critical in that the ES cells in which the locus was targeted are derived from a 129/Ola strain of mice and there is an uncharacterised defect in DAP12 signalling in 129 strains of mice⁸⁶.

To determine the role of TREM-1/3 on sepsis survival, we compared the response of TREM-1/3+/+ and TREM-1/3−/− mice (both from parallel breedings to give homogenous 70% C57BL/6/30% 129Ola background) to a CLP sepsis challenge; survival was monitored for 14 days. Studies were limited to male mice to avoid the confounding effects of the estrous cycle on sepsis survival. We found that TREM-1/3−/− mice were resistant to CLP as compared to WT (FIG. 12).

Our data demonstrate that abrogation of TREM-1/3 signalling provides protection in this murine model of CLP, substantiating our previously published data that blockade of TREM-1 signalling is beneficial in CLP. The 70% C57BL/6 mice are more resistant to CLP than their WT counterparts indicating that modulation of TREM signalling may be beneficial in sepsis.

Once these knockout mice are on a pure C57BL/6 background, we expect the phenotypic protection of the TREM-1/3−/− to be even more substantial. Strains of mice in which single receptors have been deleted can be generated and further analysed.

### EXAMPLE 6

### Establishing a Murine Model of Pulmonary Sepsis

Previously we have shown that blockade of TREM-1 can improve survival after cecal ligation and puncture (CLP) induced sepsis. CLP is a clinically relevant model of abdominal sepsis and recapitulates the pathogenesis of a polymicrobial gram-negative sepsis secondary to endogenous bacteria. However, sepsis of abdominal origin comprises only a small fraction of the clinical disease. In the US, greater than 50% of clinical sepsis extends from a primary pulmonary infection with either community acquired or nosocomial pathogens. Moreover, human TREM-1 is strongly expressed in alveolar macrophages³⁷ and may have an important function in host responses to pulmonary infections. To better model the clinical condition, we have adopted models of pulmonary sepsis initiated by infection with single clinically relevant pathogens. Previously, Drs Coopersmith and Hotchkiss have published a model of gram-negative sepsis from Pseudomonas aeruginosa pneumonia⁸⁷⁻⁸⁹. Recently, this model has been extended to gram-positive sepsis from Streptococcus pneumoniae pneumonia. In both models, bacterial inoculum has been titrated to give 90% mortality in WT mice. FIG. 13 shows the survival curves for these two models of sepsis (left panel is Streptococcus pneumoniae model and right panel is Pseudomonas aeruginosa model)

### EXAMPLE 7

### Human TREM-1-Ligand(s) is Expressed on Activated Neutrophils

Until now, efforts to identify the ligand for TREM-1 in several laboratories have been ineffective. In the inventors' view, this is likely due to a low affinity receptor-ligand interaction coupled with a rapid off rate. This phenomenon has been described in the innate immunity literature. For example, the NK receptor NKG2D has multiple ligands with affinities varying from micromolar to nanomolar⁹⁰. To identify the low affinity ligands of immunoreceptors, the inventors have developed a new approach. Various tetrameric and multimeric constructs have been designed to create higher receptor-ligand affinities through polyvalency and a more favorable on off rate⁹¹⁻⁹⁴. To this end, we generated a tetrameric TREM-1 construct. A cDNA encoding human TREM-1 ectodomain was cloned into a bacterial expression vector Pet 28 (kindly provided by Daved Fremont, Washington University School of Medicine), which incorporates a BirA tag and a 6-histidines tag on the carboxy terminus of the protein of interest (FIG. 14). The BirA sequence is efficiently biotinylated with recombinant biotin ligase. The polyhistidine tag can be used to purify the recombinant protein by nickel sepharose chromatography. This protein (TREM-1 ectodomain-BirA-6H) was purified from bacterial inclusion bodies, refolded and then isolated utilizing FPLC. Subsequently, the protein was biotinylated. Unincorporated biotin was removed by FLPC. Biotinylated TREM-1 was then incubated with streptavidin coupled to phycoerythrin (PE). As streptavidin contains 4 distinct high affinity (10⁻¹²M) biotin-binding sites, biotinylated TREM-1 ectodomain and PE-streptavidin form PE-labeled tetramers. The resultant molecule, hTREM-1 tetramer, has four hTREM-1 ectodomains displayed on the central streptavidin molecule coupled to PE.

Using this PE-labeled tetrameric TREM-1, we screened existing cell lines (over 30 human and mouse lines were examined) and peripheral blood mononuclear cells (PBMC), mouse lymph node, spleen and peritoneal cells by flow cytometry. Because our preliminary data indicated that TREM-1 was critical in amplifying inflammatory signals, the inventors examined cells obtained from patients with sepsis in the Intensive Care Units (ICU) at Barnes Jewish Hospital. After obtaining approval from the Institutional Review Board to screen the patient samples, they identified appropriate ICU patients and the ICU staff collected blood. Neutrophils were isolated using ficoll gradient followed by dextran enrichment⁹⁵. Great care was taken to perform the isolation rapidly, with minimal centrifugation, and cells were kept at 4° C. to avoid artifactual activation⁹⁶. Patients were selected based on the presence of suspected infection and the requirement for vasopressors to support blood pressure. Blood was collected and the neutrophils were then utilized for further analysis. Concurrently, control neutrophils from ambulatory volunteers were obtained and processed in parallel with patient samples for binding experiments. The hTREM-1 tetrameric construct bound to a subset of neutrophils from septic patients but not neutrophils from healthy volunteers (FIG. 14). These data suggest that the putative TREM-1 ligand is expressed on neutrophils in septic patients. To confirm the specificity of hTREM-1 tetramer binding, a control tetramer (CD69) and SA-PE alone were used in binding assays and both failed to bind to human neutrophils obtained from patients with sepsis (FIG. 14).

The subpopulation of neutrophils that bound hTREM-1 tetramer was characterised using mAbs against cell lineage markers. This subpopulation was CD56-, CD3-, CD19-, and CD16^{high} consistent with a neutrophilic pattern of receptors. Further analysis revealed that this subpopulation of neutrophils was positive for CD11b, CD10, CD66b, CD55, and CD11c all markers known to be expressed on circulating mature neutrophils⁹⁶ (FIG. 15). This population was notably CD35 (complement receptor 1) positive as well indicating that these cells are mature segmented neutrophils and not immature granulocytes released early from the bone marrow in response to stress, as the CD35 receptor has been reported to be an antigen which appears at the band and segment stage of neutrophil development⁹⁷. It has been previously reported that CD16 levels are abnormally low in setting of inflammation and infection⁹⁸. In agreement with these studies, CD16 (Fcγ receptor III) levels were decreased in the septic patients' neutrophils as compared to controls (FIG. 14). Interestingly, the percentage of neutrophils positive for the TREM-1 ligand varied between patients from approximately 25% up to 90%. The etiology of this variability at this time is unclear but may represent different states of neutrophil activation, genetic differences between the patients or variable post-translational protein modifications. Identification of the TREM-1 ligand allows further analysis of genetic variability. Such variability has been noted in both the incidence and outcomes in sepsis.

### EXAMPLE 8

### PMA/Ionomycin Upregulates Putative hTREM-1 Ligand

To assess whether neutrophils treated with the in vitro stimulation would upregulate the putative ligand, control neutrophils were examined.

Blood was collected from septic patients in the intensive care unit at Barnes Hospital in accordance with the Human Studies Committee Protocol. Neutrophils were isolated by standard protocol. Briefly, blood was diluted with 2 parts PBS and then overlaid on 15 ml of ficoll in a 50 ml conical. This tube was spun at 1400 rpm for 30 minutes. Neutrophils mixed with red blood cells were then further separated using a 3% dextran solution. The neutrophil enriched layer was then collected and rbcs were subjected to hypotonic lysis using 0.2% NaCl for 30 seconds followed by an equal volume 1.6% NaCl. Neutrophils were then pelleted and resuspended in cold PBS.

Following isolation from the blood, the neutrophils were treated with a variety of stimuli including fMLP, TNF-α, LPS, IL-1, and PMA/Ionomycin. These agents were chosen because they all stimulate human neutrophils causing some preferential degranulation of neutrophil granules. Neutrophils have several unique types of granules, including specific, azurophilic, gelatinase, and secretory vesicles. Each type of granule contains characteristic proteins. Once the granule is exocytosed, the membrane of mobilized granule remains part of the plasma membrane, thus displaying molecules previously intracellular.⁹⁹ This is a mechanism for the neutrophil to display new receptors rapidly upon stimulation. By stimulating the cells with different compounds, we hoped to ascertain whether the ligand was synthesised de novo upon activation or preformed in granules.

When control neutrophils were stimulated with the above compounds, only cells treated with PMA/ionomycin bound the human TREM-1 (FIG. 14). It has been shown previously that PMA/ionomycin provides such strong activation (through calcium flux and protein kinase C activation) that more than 50% of the neutrophil's total granular contents are exocytosed¹⁰⁰. Under these conditions, almost the entire neutrophil population became positive for hTREM-1 tetramer binding. This binding was initially detected after only 3 minutes of exposure to PMA/ionomycin. These data indicate that at least some portion of the hTREM-1 ligand is preformed and following the appropriate stimulation, the neutrophils translocate the ligand to the surface where it is then available for tetramer binding. Whether some component of this upregulation is driven at the translational level is unclear as maximal hTREM-1 tetramer binding occurred at 30-45 min after PMA/Ionomycin exposure. At this time we cannot make any inferences regarding the cytoplasmic location of the preformed ligand. Together these data indicate that the ligand for hTREM-1 is expressed on a subpopulation of neutrophils in patients with sepsis and neutrophils activated with PMA/ionomycin. These data are consistent with a role for TREM-1 signalling in inflammation and the evolution of sepsis. Indeed, one would expect that since the TREM-1 receptor is expressed constitutively on neutrophils and monocytes, that it would be ligand expression which is dynamic in the setting of inflammation. Regulation of ligand expression could play a critical role in the evolution of sepsis following the initial inflammatory trigger.

### EXAMPLE 9

### Generation of TREM-1-Ligand Blocking Antibodies

To identify the putative ligand, anti-human neutrophil antibodies were generated. Rats were immunised with ligand positive neutrophils isolated from septic patients. After three rounds of immunisation, the rats were sacrificed and their spleens were fused with mouse SP2/0 mouse myeloma cells. The resulting hybridomas were screened for the production of antibodies that:

a) bound to PMA/Ionomycin stimulated neutrophils or septic patient neutrophils; b) did not extensively bind to control neutrophils; c) did not bind TREM-1-transfected cells; d) abrogated TREM-1 tetramer binding to activated neutrophils. Following this screening procedure, a mAb (IgG2a), designated R33 was identified. The antigen recognized by R33 was upregulated on neutrophils from septic patients and neutrophils pretreated with PMA/ionomycin. Importantly, preincubation of neutrophils from septic patients with mAb R33 abrogates TREM-1 tetramer binding while preincubation with an isotype matched control mAb did not interfere with tetramer binding (FIG. 16). Based on these data, the inventors conclude that the R33 antigen is a ligand for TREM-1.

### EXAMPLE 10

### Construction of a cDNA Expression Library made from Septic Patient Neutrophils and use of this Library to Identify the TREM-1 Ligand

Total cellular RNA from the buffy coats of septic patients in the ICU who met criteria for sepsis was prepared. The majority of these patients were screened for R33 antigen binding activity as well as TREM-1 tetramer binding. (FIG. 22 shows that TREM-1 tetramer binds to septic patients neutrophils and not to resting neutrophils.)

To purify the RNA we utilized a protocol previously validated in Dr. Perren Cobbs laboratory as part of the Inflammation and Host Response to Injury Large Scale Collaborative Research Program¹⁰¹. Briefly, samples were processed within two hours of generation. Blood was spun at 900×g for 10 minutes without brakes. Serum was then removed and stored at −80° C. The cell pellet was resuspended in EL buffer (InVitrogen) and incubated on ice for 15 minutes. Following this incubation the cells were collected and this step was repeated. Once the sample was free of red blood cells, RNA storage buffer was added and samples were frozen at −80° C. The total cellular RNA was then isolated using the RNAEASY kit from InVitrogen. The quality of the RNA was assessed by the Agilent bioanalyzer. Once adequate amounts of high quality RNA were purified, the samples were pooled and a custom nonamplified cDNA library was constructed by OpenBiosystems for our use.

We then transfected the purified cDNA into mammalian 293 cells and sorted these cells using a fluorescent cell sorter. We collected approximately 100,000 cells out of 10 million sorted (FIG. 17, panel A). We isolated the plasmid DNA from the cells using Hirt buffer. This DNA was transformed into E. coli. The transformed bacteria were plated using ampicillin selection. Once colonies were visible, the colonies were replica plated. We collected and purified plasmid from 24 individual plates, storing their replicas at 4° C. These plasmid pools were transfected into individual wells of 293 cells using lipofectamin (InVitrogen). After 24 hours, cells were harvested from each well and stained with mAb R33 and the appropriate secondary conjugated antibody. The cells were then subjected to FACS analysis (FIG. 17). Two positives plates, F (FIG. 17, panel B) and H were identified. Plate F had approximately 149 individual colonies. These colonies were divided into 4 pools and the plasmid DNA was isolated. Following transfection of the DNA into 293 cells, another round of screening by FACS staining was performed. Through this process the R33 antigen was narrowed down eventually to a single colony (FIG. 17, panel C). This was found to express CD177, a molecule which is expressed on neutrophils and a subset of monocytes.

The amino acid sequence of this molecule is shown in FIG. 18. Here it can be seen that the molecule has a GPI anchor. It also has an extracellular portion that is involved in binding to the TREM-1 receptor. The cDNA sequence is shown in FIG. 21A. GPI linked proteins are often shed from a cell surface and found as soluble proteins in plasma and serum. This has been shown to be the case for members of this protein family. For example Klippel et al (Blood, 100, No 7, 2441-2448 [2002]) report this phenomenon for PRV-1.

### EXAMPLE 11

### Generation of a Soluble form of the TREM-1 Ligand and Analysis Thereof

A soluble form of the ligand can be generated and is useful in order to perform binding assays on cells expressing TREM-1. This can be achieved using a construct in our laboratory which encodes a mutated form (does not bind to Fc receptors) of the Fc portion of IgG. The TREM-1 ligand gene can be fused in frame with the Fc. This plasmid can be transfected into mammalian cells. The protein product will be secreted into the media forming dimers via the Fc interaction. The resulting protein product will be a Fc fusion protein with two ligand heads. Supernatant from cells secreting this molecule can be collected and the molecule can be purified using a protein G column. This construct is useful for assessing the binding of receptor and ligand in both directions, i.e. soluble TREM-1 binds surface expressed R33 antigen and soluble R33 antigen binds surface expressed TREM-1. Our laboratory has used this strategy to characterize several ligand receptor interactions in the past^{13,14,114}.

The soluble TREM-1 ligand Fc protein can then be incubated with both cells expressing TREM-1 naturally (neutrophils and monocytes) as well as cells transfected with TREM-1 encoding plasmids. Binding of the Fc fusion protein can be detected using an anti human Fc conjugated to PE and FACS analysis. The converse experiment can also be performed in which the ability of the human TREM-1 tetramer to bind a cell line transfected with the TREM-1 ligand can be assessed. The TREM-1 ligand can be amplified from the cDNA library plasmid and subcloned into pcDNA3 vector. This vector contains a neomycin selection allowing for the production of stable mammalian transfectants. The plasmid can be transfected into 293 cells and placed under antibiotic selection. Resistant cells can then be analyzed in FACS for staining with the R33 antibody. High expressing stable transfectants can be cloned and then used in binding assays with the TREM-1 tetramer molecule as well as the TREM-1 Fc molecule previously made in our laboratory.

A T cell hybridoma reporter cell line has been constructed which expresses the TREM-1 molecule fused to the cytoplasmic region of CD3ζ. If the TREM-1 molecule is engaged in a functional way, ZAP70 is recruited to the CD3ζ and a series of intracellular phosphorylation events lead to activation of PLCg and increased intracellular calcium. The reporter cell contains a plasmid encoding the NFAT promoter fused to a sequence encoding green fluorescent protein (GFP). NFAT is activated by intracellular calcium mobilization. This allows one to co-incubate the TREM-1 expressing GFP reporter with a putative ligand and then analyze the cells for GFP expression by FACS. Our laboratory and others have used this system to ascertain the biological relevance of other receptor ligand interactions¹¹⁵. This functional assay system can be used as another measure of the biological relevance of the TREM-1/TREM-1 ligand interaction.

The soluble TREM-1 ligand molecule as well as an irrelevant control Fc fusion protein can be incubated with freshly isolated human neutrophils and monocytes. IL-1, IL-8 and MPO activity will be measured in the neutrophils as surrogate markers of inflammation. In addition the effect of R33 antigen binding on neutrophil phagocytosis will also be assessed. In the monocytes, the secretion of TNFα, IL-8, and MCP-1 can be measured to ascertain the effects of TREM-1 engagement on these molecules. We expect engagement to result in the secretion of these proinflammatory cytokines.

Wild type and TREM1/3 deficient mice can be used to assess the impact of soluble TREM-1 ligand on murine sepsis. Survival, serum cytokine production, peritoneal infiltration and local and systemic bacterial load can be assessed in these mice. We predict that excess TREM-1 ligand in the knockout mice should have no impact on survival whereas excess TREM-1 ligand, if stimulatory should increase cytokine production and mortality in the wild type mice.

We expect binding of the TREM-1 ligand to trigger proinflammatory cytokine production. In vivo we expect that administration of soluble TREM-1 ligand will result in increased cytokine production and increased mortality in wild type mice following CLP while the knockout mice should be unaffected by this molecule.

### EXAMPLE 12

### TREM-1 Ligand as a Marker of Sepsis

Patients included in the study were 26 newly admitted patients who presented with clinically suspected infection and fulfilled at least two criteria of the Systemic Inflammatory Response Syndrome (SIRS) [Bone R C, Sibbald W J and Sprung C L. The ACCP-SCCM consensus conference on sepsis and organ failure. Chest 1992;101:1481-3]. The patients were retrospectively classified as follows: 12 with sepsis and 14 with SIRS. Four healthy individuals were included as controls. TREM-1 ligand expression was evaluated at two time-points: 1) acute phase, immediately after admission into the ICU (temperature>38° C., heart rate>90/min, WBC>12×10⁹/l); and 2) recovery, corresponding to the time of clinical discharge (normalization of the above clinical parameters). Clinical characteristics at inclusion did not differ significantly between patients with sepsis and those without: male n° 8 (66%) and 9 (69%); age 48.6 and 58.5 in sepsis and SIRS respectively.

Bloodstream infections were microbiologically proven in all 12 patients with sepsis (5 Gram⁺, 5 Gram⁻, 1 multiple infections, 1 C. albicans). TREM-1 ligand expression was detected only in patients with sepsis but not in those with SIRS (FIG. 24A). Peripheral granulocytes from healthy subjects did not express detectable levels of TREM-1 ligand (FIG. 24A). No correlation was observed between levels of TREM-1 ligand expression and the microbial strain isolated from the bloodstream, or any other clinical or biological feature. We further evaluated the relationship between levels of expression of TREM-1 ligand and the clinical status of sepsis patients. In all the sepsis patients analyzed, the levels of expression of TREM-1 ligand decreased at the time of discharge from the ICU (FIG. 24B). In one patient, the second determination of TREM-1 ligand could not be performed because the patient died from septic shock. In two more patients, TREM-1 ligand expression could not be detected at the time of admission into the ICU, despite documented systemic bacterial infection. This might have been due to the fact that inadequate blood samples with high cell mortality were delivered to the laboratory.

Our results indicate that TREM-1 ligand expression is exclusively detected on peripheral neutrophils from patients with sepsis but not with SIRS of non-microbial origin, therefore representing a useful marker of sepsis. Measurement of plasma levels of soluble TREM-1 has also shown its diagnostic accuracy in distinguishing sepsis from SIRS [Gibot S, Kolopp-Sarda M N, Bene M C, et al. Ann Intern Med 2004; 141:9-15]. Indeed, advances in sepsis research require better markers than the ones available to delineate more homogenous subsets of patients within a highly heterogeneous group of critically ill patients, and to identify patients having the particular biological abnormality that a proposed therapy will target. Our data suggest that TREM-1 ligand might represent a useful diagnostic marker to predict the presence and severity of sepsis providing information in establishing a diagnosis to identify a patient who has the disease and therefore might respond to a particular therapy; quantifying the severity of sepsis to identify patients who are more likely to experience a beneficial outcome; measuring the response to therapy to determine how a patient is responding to an intervention.

Moreover TREM-1 ligand is an important mediator in sepsis and it is specifically expressed in patients with sepsis. Since intervention must not only be targeted to TREM-1 but it must be given at the appropriate time, the analysis of the expression of TREM-1 ligand during the evolution of the inflammatory response during sepsis is of fundamental importance in effective therapies for sepsis.

### EXAMPLE 13

### First Example Showing how Screening for Compounds that Prevent/Reduce the Binding of a TREM-1 Ligand to its Receptor can be Performed

293 cells alone or 293 cells transfected with murine CD177 were preincubated with different concentrations of test compounds for 30 minutes on ice and then incubated with soluble murine TREM-1 molecule (100 ug/ml) for 45 minutes on ice, cells were then washed with FACS buffer (PBS, 2% BCS), incubated with anti human FC biotin for 20 minutes on ice, washed once with FACS buffer, and incubated with streptavidin APC for 20 minutes, following a wash with FACS buffer, the cells were immediately analyzed. Dead cells were excluded. A shift of the histogram to the left indicates that the test compound is inhibiting binding of the TREM-1 molecule to CD177.

### EXAMPLE 14

### Second Example Showing how Screening for Compounds that Prevent/Reduce the Binding of a TREM-1 Ligand to its Receptor can be Performed

A T cell hybridoma reporter cell line has been constructed which expresses the TREM-1 molecule fused to the cytoplasmic region of CD3ζ. If the TREM-1 molecule is engaged in a functional way, ZAP70 is recruited to the CD3ζ and a series of intracellular phosphorylation events lead to activation of PLCg and increased intracellular calcium. The reporter cell contains a plasmid encoding the NFAT promoter fused to a sequence encoding green fluorescent protein (GFP). NFAT is activated by intracellular calcium mobilization. This allows one to co-incubate the TREM-1 expressing GFP reporter with CD177—either in a soluble form or expressed by a transfected cell line—in the presence or absence of different concentrations of test compounds and then analyze the cells for GFP expression by FACS. Inhibition of activation of the TREM-1 reporter cell line by CD177 indicates that the test compound binds to TREM-1. The above system can be modified by using different reporter systems, such as lacZ.

### EXAMPLE 15

### Example Showing how Diagnostic Screening of Patients for Sepsis could be Performed Based Upon the Identification of the TREM-1 Ligand

A TREM-1 ligand (e.g. CD177) or a TREM-1 ligand binding portion thereof can be obtained in pure form.

This can then be inoculated into an animal and used to generate a series of hybridomas producing monoclonal antibodies.

The antibodies can then be screened using the screening procedures of the present invention in order to identify ones that block or reduce the binding of the TREM-1 ligand to a TREM-1 receptor.

Such antibodies can then be used in diagnostic tests to diagnose sepsis (especially of microbial origin, e.g. of bacterial or fungal origin).

If desired a control may be used based upon neutrophils or monocytes from a healthy patient.

If the antibodies bind to the patient thought to be at risk of sepsis to a significantly higher degree than the control, then this is an indicator of sepsis.

This test can also distinguish between sepsis of microbial origin and non-microbial derived SIRS. In the latter case (unlike the former) there is no substantial binding of the antibodies to the neutrophils or monocytes obtained from a patient.

### EXAMPLE 16

### Example Showing how an Antibody that Specifically Blocks the Binding of the TREM-1 Ligand to its Receptor could be Identified and Used in the Treatment of Sepsis

Monoclonal antibodies to the TREM-1 ligand can be raised and screened as discussed in Examples 13-14.

Anitibodies identified by screening as being successful in blocking the binding of TREM-1 to its receptor can then be used for further testing.

For example they can be used to see if they bind to peripheral neutrophils from patients with microbial sepsis but not with SIRS of non-microbial origin (see Example 12).

Antibodies that are successful in this test can be selected for further analysis, including safety testing and possible eventual clinical trials.

Clinical trials can be performed by comparing the results of the antibodies on a patient group with microbial sepsis with results for a patient group of non-microbial origin. The trails will be successful if there are no major side effects with either group and there is a significant improvement in the condition of the patient group with microbial sepsis, relative to the patient group with SIRS of non-microbial origin. Appropriate control groups can also be used, e.g. patients with microbial sepsis who are given a placebo, patients with SIRS of non-microbial origin who are given a placebo, a group of healthy volunteers that are given a placebo and a group of healthy volunteers that are given the antibody.

The antibodies can be provided in a form that reduces cross-reactivity. For example they can be “humanised”” or even “completely human”, as discussed earlier. They can be provided in a sterile pharmaceutical composition together with one or more substances that help extend the half life in vivo (e.g. pegylation can be used as discussed earlier). They can be administered by any appropriate route, but are preferably provided as an injectable composition.

Dosage ranges are given earlier, but can of course be optimised by the results of animal trials before administration to humans. If side effects develop at a certain dosage then the dosage should of course be reduced as appropriate.

### EXAMPLE 17

### Example of Providing Antibodies to a Non-Human TREM-1 Ligand

There are of course intra and inter species variants of CD177 and of other TREM-1 ligands (e.g. PRV-1). Antibodies to different variants can be useful in purification, diagnosis, treatments, tissue typing, comparative studies, assessments of specificity, etc.

A monoclonal antibody (R33) to CD177 expressed by humans has already been discussed.

This example illustrates the generation of antibodies to murine CD177.

Murine CD177 was identified using blast homology searches. Specific primers were generated and used to amplify the CD177 sequence from murine cDNA. The resulting fragment was subcloned into the expression vector pCDNA6. This plasmid was transfected into 293 cells and stable high expressing cells were isolated using high efficiency cell sorter. These cells were then utilized to immunize rats. Subsequently the rat lymph node was fused to SP2/0 cells and following HAT selection, individual antibody producing clones were isolated and screened for binding to the recombinant mouse CD177 molecule. Forty positive clones were identified. One of these antibodies was then purified and biotinylated (Y176) to be used to ascertain where CD177 was expressed in the mouse. Bone marrow was harvested and incubated with FcBlocking supernate. Following a 20 minute room temperature incubation, biotinylated Y176 (followed by streptavidin APC), anti CD11b fitc and anti GR1 PE was used to characterize the CD177 positive population. Examination of bone marrow revealed CD177 is expressed on inflammatory monocytes and neutrophils.

### EXAMPLE 18

### R33 (Anti-Human CD177) Blocks mTREM-1 Binding to hCD177 Transfected HEK293 Cells

HEK293 cells transfected with the human CD177 full length were analyzed by cytofluorimetric analysis, as shown in FIG. 25. The grey histogram represents staining with soluble mouse TREM1/IgG in the presence of an isotype control MAb. The dashed histogram represents staining with soluble mouse TREM1/IgG in the presence of the R33 MAb. Staining with a control soluble mouse TLT/IgG is represented by the white histogram.

The data show that the the R33 MAb specifically blocks binding of soluble mouse TREM1/IgG to CD177-transfected cells.

### EXAMPLE 19

### Mouse CD177 is Expressed on Neutrophils and Monocytes

Mouse peripheral blood was analyzed by cytofluorimetric analysis. The results are shown in FIG. 26

LEFT: The dot plot represents forwards vs size scatter of mononuclear cells in the mouse peripheral blood. Based on physical parameters, three gates were constructed that identify different subsets: a) lymphocytes, b) monocytes, c) neutrophils.

RIGHT: The three panels on the right show the staining of the cells with the Y176 MAb.

The data show that the Y176 MAb specifically recognizes its epitope on peripheral blook neutophils and monocytes but not on lymphocytes.

### EXAMPLE 20

### Murine TREM-1 Soluble Molecule Binds to 293 Cells Transfected with Murine CD177

293 cells alone or 293 cells transfected with murine CD177 were incubated with soluble murine TREM-1 molecule (100 ug/ml) for 45 minutes on ice, cells were then washed with FACS buffer (PBS, 2% BCS), incubated with anti human FC biotin for 20 minutes on ice, washed once with FACS buffer, and incubated with streptavidin APC for 20 minutes, following a wash with FACS buffer, the cells were immediately analyzed. Dead cells were excluded.

The results are shown in FIG. 27. In the histogram, murine TREM-1 soluble molecule binding to 293/murineCD177 transfected cells is shown in dashed line while murine TREM-1 soluble molecule binding to 293 only is shown in solid line.

This provides evidence that mTREM-1 binds mCD177 expressed on 293 cells.

### LITERATURE REFERENCES

1. Beutler B. Inferences, questions and possibilities in Toll-like receptor signalling. Nature. 2004; 430:257-263.
2. Pasare C, Medzhitov R. Toll-like receptors: balancing host resistance with immune tolerance. Curr Opin Immunol. 2003; 15:677-682.
3. Akira S. Toll receptor families: structure and function. Semin Immunol. 2004; 16:1-2.
4. Takeda K, Akira S. TLR signalling pathways. Semin Immunol. 2004; 16:3-9.
5. Cohen J. The immunopathogenesis of sepsis. Nature. 2002; 420:885-891.
6. Murphy T J, Paterson H M, Mannick J A, Lederer J A. Injury, sepsis, and the regulation of Toll-like receptor responses. J Leukoc Biol. 2004; 75:400-407.
7. Beutz M A, Abraham E. Community-acquired pneumonia and sepsis. Clin Chest Med. 2005; 26:19-28.
8. Ayala A, Chung C S, Grutkoski P S, Song G Y. Mechanisms of immune resolution. Crit Care Med. 2003; 31:5558-571.
9. Hotchkiss R S, Karl I E. The pathophysiology and treatment of sepsis. N Engl J Med. 2003; 348:138-150.
10. Minnich D J, Moldawer L L. Anti-cytokine and anti-inflammatory therapies for the treatment of severe sepsis: progress and pitfalls. Proc Nutr Soc. 2004; 63:437-441.
11. Opal S M, Cross A S. Clinical trials for severe sepsis. Past failures, and future hopes. Infect Dis Clin North Am. 1999; 13:285-297, vii.
12. Colonna M. TREMs in the immune system and beyond. Nat Rev Immunol. 2003; 3:445-453.
13. Bouchon A, Dietrich J, Colonna M. Cutting edge: inflammatory responses can be triggered by TREM-1, a novel receptor expressed on neutrophils and monocytes. J Immunol. 2000; 164:4991-4995.
14. Bouchon A, Facchetti F, Weigand M A, Colonna M. TREM-1 amplifies inflammation and is a crucial mediator of septic shock. Nature. 2001; 410:1103-1107.
15. Bleharski J R, Kiessler V, Buonsanti C, et al. A role for triggering receptor expressed on myeloid cells-1 in host defense during the early-induced and adaptive phases of the immune response. J Immunol. 2003; 170:3812-3818.
16. Netea M G, Azam T, Ferwerda G, Girardin S E, Kim S H, Dinarello C A. Triggering receptor expressed on myeloid cells-1 (TREM-1) amplifies the signals induced by the NACHT-LRR (NLR) pattern recognition receptors. J Leukoc Biol. 2006.
17. Klesney-Tait J, Turnbull I R, Colonna M. The TREM receptor family and signal integration. Nat Immunol. 2006; In press.
18. Daws M R, Lanier L L, Seaman W E, Ryan J C. Cloning and characterization of a novel mouse myeloid DAP12-associated receptor family. Eur J Immunol. 2001; 31:783-791.
19. Chung D H, Seaman W E, Daws M R. Characterization of TREM-3, an activating receptor on mouse macrophages: definition of a family of single Ig domain receptors on mouse chromosome 17. Eur J Immunol. 2002; 32:59-66.
20. Allcock R J, Barrow A D, Forbes S, Beck S, Trowsdale J. The human TREM gene cluster at 6p21.1 encodes both activating and inhibitory single IgV domain receptors and includes NKp44. Eur J Immunol. 2003; 33:567-577.
21. Vivier E, Nunes J A, Vely F. Natural killer cell signalling pathways. Science. 2004; 306:1517-1519.
22. Lanier L L. Natural killer cell receptor signalling. Curr Opin Immunol. 2003; 15:308-314.
23. McVicar D W, Burshtyn D N. Intracellular signalling by the killer immunoglobulin-like receptors and Ly49. Sci STKE. 2001; 2001:RE1.
24. Washington A V, Schubert R L, Quigley L, et al. A TREM family member, TLT-1, is found exclusively in the alpha-granules of megakaryocytes and platelets. Blood. 2004; 104:1042-1047.
25. Washington A V, Quigley L, McVicar D W. Initial characterization of TREM-like transcript (TLT)-1: a putative inhibitory receptor within the TREM cluster. Blood. 2002; 100:3822-3824.
26. King R G, Herrin B R, Justement L B. Trem-like transcript 2 is expressed on cells of the myeloid/granuloid and B lymphoid lineage and is up-regulated in response to inflammation. J Immunol. 2006; 176:6012-6021.
27. Cantoni C, Bottino C, Vitale M, et al. NKp44, a triggering receptor involved in tumor cell lysis by activated human natural killer cells, is a novel member of the immunoglobulin superfamily. J Exp Med. 1999; 189:787-796.
28. Jackson D G, Hart D N, Starling G, Bell J I. Molecular cloning of a novel member of the immunoglobulin gene superfamily homologous to the polymeric immunoglobulin receptor. Eur J Immunol. 1992; 22:1157-1163.
29. Green B J, Clark G J, Hart D N. The CMRF-35 mAb recognizes a second leukocyte membrane molecule with a domain similar to the poly Ig receptor. Int Immunol. 1998; 10:891-899.
30. Speckman R A, Wright Daw J A, Helms C, et al. Novel immunoglobulin superfamily gene cluster, mapping to a region of human chromosome 17q25, linked to psoriasis susceptibility. Hum Genet. 2003; 112:34-41.
31. Chung D H, Humphrey M B, Nakamura M C, Ginzinger D G, Seaman W E, Daws M R. CMRF-35-like molecule-1, a novel mouse myeloid receptor, can inhibit osteoclast formation. J Immunol. 2003; 171:6541-6548.
32. Raghavan M, Bjorkman P J. Fc receptors and their interactions with immunoglobulins. Annu Rev Cell Dev Biol. 1996; 12:181-220.
33. Paloneva J, Manninen T, Christman G, et al. Mutations in Two Genes Encoding Different Subunits of a Receptor Signalling Complex Result in an Identical Disease Phenotype. Am J Hum Genet. 2002; 71:3.
34. Humphrey M B, Daws M R, Spusta S C, et al. TREM2, a DAP12-associated receptor, regulates osteoclast differentiation and function. J Bone Miner Res. 2006; 21:237-245.
35. Turnbull I R, Gilfillan S, Cella M, et al. Cutting edge: TREM-2 attenuates macrophage activation. J Immunol. 2006; 177:3520-3524.
36. Hamerman J A, Jarjoura J R, Humphrey M B, Nakamura M C, Seaman W E, Lanier L L. Cutting edge: inhibition of TLR and FcR responses in macrophages by triggering receptor expressed on myeloid cells (TREM)-2 and DAP12. J Immunol. 2006; 177:2051-2055.
37. Colonna M, Facchetti F. TREM-1 (triggering receptor expressed on myeloid cells): a new player in acute inflammatory responses. J Infect Dis. 2003; 187 Suppl 2:S397-401.
38. Lucas M, Daniel L, Tomasello E, et al. Massive inflammatory syndrome and lymphocytic immunodeficiency in KARAP/DAP12-transgenic mice. Eur J Immunol. 2002; 32:2653-2663.
39. Nochi H, Aoki N, Oikawa K, et al. Modulation of hepatic granulomatous responses by transgene expression of DAP12 or TREM-1-Ig molecules. Am J Pathol. 2003; 162:1191-1201.
40. Gibot S, Cravoisy A, Kolopp-Sarda M N, et al. Time-course of sTREM (soluble triggering receptor expressed on myeloid cells)-1, procalcitonin, and C-reactive protein plasma concentrations during sepsis. Crit Care Med. 2005; 33:792-796.
41. Gibot S, Kolopp-Sarda M N, Bene M C, et al. A soluble form of the triggering receptor expressed on myeloid cells-1 modulates the inflammatory response in murine sepsis. J Exp Med. 2004; 200:1419-1426.
42. Gibot S, Cravoisy A, Levy B, Bene M C, Faure G, Bollaert P E. Soluble triggering receptor expressed on myeloid cells and the diagnosis of pneumonia. N Engl J Med. 2004; 350:451-458.
43. Gingras M C, Lapillonne H, Margolin J F. TREM-1, MDL-1, and DAP12 expression is associated with a mature stage of myeloid development. Mol Immunol. 2002; 38:817-824.
44. Begum N A, Ishii K, Kurita-Taniguchi M, et al. Mycobacterium bovis BCG cell wall-specific differentially expressed genes identified by differential display and cDNA subtraction in human macrophages. Infect Immun. 2004; 72:937-948.
45. Bourke E, Cassetti A, Villa A, Fadlon E, Colotta F, Mantovani A. IL-1 beta scavenging by the type II IL-1 decoy receptor in human neutrophils. J Immunol. 2003; 170:5999-6005.
46. Muller B, Peri G, Doni A, et al. High circulating levels of the IL-1 type II decoy receptor in critically ill patients with sepsis: association of high decoy receptor levels with glucocorticoid administration. J Leukoc Biol. 2002; 72:643-649.
47. McGeehan G M, Becherer J D, Bast R C, Jr., et al. Regulation of tumour necrosis factor-alpha processing by a metalloproteinase inhibitor. Nature. 1994; 370:558-561.
48. Mohler K M, Sleath P R, Fitzner J N, et al. Protection against a lethal dose of endotoxin by an inhibitor of tumour necrosis factor processing. Nature. 1994; 370:218-220.
49. Gearing A J, Beckett P, Christodoulou M, et al. Processing of tumour necrosis factor-alpha precursor by metalloproteinases. Nature. 1994; 370:555-557.
50. Mullberg J, Durie F H, Otten-Evans C, et al. A metalloprotease inhibitor blocks shedding of the IL-6 receptor and the p60 TNF receptor. J Immunol. 1995; 155:5198-5205.
51. Walcheck B, Kahn J, Fisher J M, et al. Neutrophil rolling altered by inhibition of L-selectin shedding in vitro. Nature. 1996; 380:720-723.
52. Bouchon A, Hernandez-Munain C, Cella M, Colonna M. A DAP12-mediated pathway regulates expression of CC chemokine receptor 7 and maturation of human dendritic cells. J Exp Med. 2001; 194:1111-1122.
53. Hazeki K, Masuda N, Funami K, et al. Toll-like receptor-mediated tyrosine phosphorylation of paxillin via MyD88-dependent and -independent pathways. Eur J Immunol. 2003; 33:740-747.
54. Murphy L O, Smith S, Chen R H, Fingar D C, Blenis J. Molecular interpretation of ERK signal duration by immediate early gene products. Nat Cell Biol. 2002; 4:556-564.
55. Assoian R K. Common sense signalling. Nat Cell Biol. 2002; 4:E187.
56. Hogan P G, Chen L, Nardone J, Rao A. Transcriptional regulation by calcium, calcineurin, and NFAT. Genes Dev. 2003; 17:2205-2232.
57. Crabtree G R, Olson E N. NFAT signalling: choreographing the social lives of cells. Cell. 2002; 109 Suppl: S67-79.
58. Koga T, lnui M, Inoue K, et al. Costimulatory signals mediated by the ITAM motif cooperate with RANKL for bone homeostasis. Nature. 2004; 428:758-763.
59. Ley K. Integration of inflammatory signals by rolling neutrophils. Immunol Rev. 2002; 186:8-18.
60. Williams M A, Solomkin J S. Integrin-mediated signalling in human neutrophil functioning. J Leukoc Biol. 1999; 65:725-736.
61. Laudanna C, Kim J Y, Constantin G, Butcher E. Rapid leukocyte integrin activation by chemokines. Immunol Rev. 2002; 186:37-46.
62. Lanier L L, Bakker A B. The ITAM-bearing transmembrane adaptor DAP12 in lymphoid and myeloid cell function. Immunol Today. 2000; 21:611-614.
63. Hamerman J A, Tchao N K, Lowell C A, Lanier L L. Enhanced Toll-like receptor responses in the absence of signalling adaptor DAP12. Nat Immunol. 2005; 6:579-586.
64. Turnbull I R, McDunn J E, Takai T, Townsend R R, Cobb J P, Colonna M. DAP12 (KARAP) amplifies inflammation and increases mortality from endotoxemia and septic peritonitis. J Exp Med. 2005; 202:363-369.
65. Hamerman J A, Lanier L L. Inhibition of immune responses by ITAM-bearing receptors. Sci STKE. 2006; 2006:re1.
66. Turnbull I R, Colonna M. Activating and inhibitory functions of DAP12. Nat Rev Immunol. 2006; in press.
67. Beutler B, Milsark I W, Cerami A C. Passive immunization against cachectin/tumor necrosis factor protects mice from lethal effect of endotoxin. Science. 1985; 229:869-871.
68. Beutler B A, Milsark I W, Cerami A. Cachectin/tumor necrosis factor: production, distribution, and metabolic fate in vivo. J Immunol. 1985; 135:3972-3977.
69. Newcomb D, Bolgos G, Green L, Remick D G. Antibiotic treatment influences outcome in murine sepsis: mediators of increased morbidity. Shock. 1998; 10:110-117.
70. Unlu M, Morgan M E, Minden J S. Difference gel electrophoresis: a single gel method for detecting changes in protein extracts. Electrophoresis. 1997; 18:2071-2077.
71. Tu G F, De Jong F, Apostolopoulos J, et al. Effect of acute inflammation on rat apolipoprotein mRNA levels. Inflammation. 1987; 11:241-251.
72. Gabay C, Kushner I. Acute-phase proteins and other systemic responses to inflammation. N Engl J Med. 1999; 340:448-454.
73. Gervois P, Kleemann R, Pilon A, et al. Global suppression of IL-6-induced acute phase response gene expression after chronic in vivo treatment with the peroxisome proliferator-activated receptor-alpha activator fenofibrate. J Biol Chem. 2004; 279:16154-16160.
74. Niessen R W, Lamping R J, Jansen P M, et al. Antithrombin acts as a negative acute phase protein as established with studies on HepG2 cells and in baboons. Thromb Haemost. 1997; 78:1088-1092.
75. Mounzer K C, Moncure M, Smith Y R, Dinubile M J. Relationship of admission plasma gelsolin levels to clinical outcomes in patients after major trauma. Am J Respir Crit Care Med. 1999; 160:1673-1681.
76. Lew A M, Maloy W L, Coligan J E. Characteristics of the expression of the murine soluble class I molecule (Q10). J Immunol. 1986; 136:254-258.
77. Dietrich J, Cella M, Seiffert M, Buhring H J, Colonna M. Cutting edge: signal-regulatory protein beta 1 is a DAP12-associated activating receptor expressed in myeloid cells. J Immunol. 2000; 164:9-12.
78. Tomasello E, Cant C, Buhring H J, et al. Association of signal-regulatory proteins beta with KARAP/DAP-12. Eur J Immunol. 2000; 30:2147-2156.
79. Barclay A N, Wright G J, Brooke G, Brown M H. CD200 and membrane protein interactions in the control of myeloid cells. Trends Immunol. 2002; 23:285-290.
Wright G J, Cherwinski H, Foster-Cuevas M, et al. Characterization of the CD200 receptor family in mice and humans and their interactions with CD200. J Immunol. 2003; 171:3034-3046.
81. Gorczynski R M, Chen Z, Clark D A, et al. Structural and functional heterogeneity in the CD200R family of immunoregulatory molecules and their expression at the feto-maternal interface. Am J Reprod Immunol. 2004; 52:147-163.
82. Gorczynski R, Chen Z, Kai Y, Lee L, Wong S, Marsden P A. CD200 is a ligand for all members of the CD200R family of immunoregulatory molecules. J Immunol. 2004; 172:7744-7749.
83. Aguilar H, Alvarez-Errico D, Garcia-Montero A C, Orfao A, Sayos J, Lopez-Botet M. Molecular characterization of a novel immune receptor restricted to the monocytic lineage. J Immunol. 2004; 173:6703-6711.
84. Bakker A B, Baker E, Sutherland G R, Phillips J H, Lanier L L. Myeloid DAP12-associating lectin (MDL)-1 is a cell surface receptor involved in the activation of myeloid cells. Proc Natl Acad Sci USA. 1999; 96:9792-9796.
85. Schwenk F, Baron U, Rajewsky K. A cre-transgenic mouse strain for the ubiquitous deletion of loxP-flanked gene segments including deletion in germ cells. Nucleic Acids Res. 1995; 23:5080-5081.
86. McVicar D W, Winkler-Pickett R, Taylor L S, et al. Aberrant DAP12 signalling in the 129 strain of mice: implications for the analysis of gene-targeted mice. J Immunol. 2002; 169:1721-1728.
87. Coopersmith C M, Stromberg P E, Davis C G, et al. Sepsis from Pseudomonas aeruginosa pneumonia decreases intestinal proliferation and induces gut epithelial cell cycle arrest. Crit Care Med. 2003; 31:1630-1637.
88. Coopersmith C M, Amiot D M, 2nd, Stromberg P E, et al. Antibiotics improve survival and alter the inflammatory profile in a murine model of sepsis from Pseudomonas aeruginosa pneumonia. Shock. 2003; 19:408-414.
89. Hotchkiss R S, Dunne W M, Swanson P E, et al. Role of apoptosis in Pseudomonas aeruginosa pneumonia. Science. 2001; 294:1783.
90. Carayannopoulos L N, Naidenko O V, Kinder J, Ho E L, Fremont D H, Yokoyama W M. Ligands for murine NKG2D display heterogeneous binding behavior. Eur J Immunol. 2002; 32:597-605.
91. Traunecker A, Luke W, Karjalainen K. Soluble CD4 molecules neutralize human immunodeficiency virus type 1. Nature. 1988; 331:84-86.
92. Altman J D, Moss P A, Goulder P J, et al. Phenotypic analysis of antigen-specific T lymphocytes. Science. 1996; 274:94-96.
93. Wright G J, Puklavec M J, Willis A C, et al. Lymphoid/neuronal cell surface OX2 glycoprotein recognizes a novel receptor on macrophages implicated in the control of their function. Immunity. 2000; 13:233-242.
94. Arthos J, Cicala C, Steenbeke T D, et al. Biochemical and biological characterization of a dodecameric CD4-Ig fusion protein: implications for therapeutic and vaccine strategies. J Biol Chem. 2002; 277:11456-11464.
95. Clark R A, Nauseef W M. Preparation and functional analysis of other human lymphoid and nonlymphoid cells. In: Coligan J E, Kruisbeek A M, Margulies D H, Shevach E M, Strober W, eds. Current Protocols in Immunology. Vol. 2: John Wiley & Sons, Inc.; 1999:7.23.21-27.23.17.
96. Elghetany M T. Surface antigen changes during normal neutrophilic development: a critical review. Blood Cells Mol Dis. 2002; 28:260-274.
97. Ahearn J M, Fearon D T. Structure and function of the complement receptors, CR1 (CD35) and CR2 (CD21). Adv Immunol. 1989; 46:183-219.
98. Kabutomori O, Iwatani Y, Koh T, Fushimi R, Amino N. Decrease in the density of IgG-Fc receptor III (CD16) on ‘toxic’ neutrophils. Acta Haematol. 1993; 89:163-164.
99. Borregaard N, Cowland J B. Granules of the human neutrophilic polymorphonuclear leukocyte. Blood. 1997; 89:3503-3521.
100. Wright D G, Bralove D A, Gallin J I. The differential mobilization of human neutrophil granules. Effects of phorbol myristate acetate and ionophore A23187. Am J Pathol. 1977; 87:237-284.
101. Calvano S E, Xiao W, Richards D R, et al. A network-based analysis of systemic inflammation in humans. Nature. 2005; 437:1032-1037.
102. Remick D G, Newcomb D E, Bolgos G L, Call D R. Comparison of the mortality and inflammatory response of two models of sepsis: lipopolysaccharide vs. cecal ligation and puncture. Shock. 2000; 13:110-116.
103. Cella M, Fujikawa K, Tassi I, et al. Differential requirements for Vav proteins in DAP10- and ITAM-mediated NK cell cytotoxicity. J Exp Med. 2004; 200:817-823.
104. Avraham H, Park S Y, Schinkmann K, Avraham S. RAFTK/Pyk2-mediated cellular signalling. Cell Signal. 2000; 12:123-133.
105. Burridge K, Wennerberg K. Rho and Rac take center stage. Cell. 2004; 116:167-179.
106. Bokoch G M. Biology of the p21-activated kinases. Annu Rev Biochem. 2003; 72:743-781.
107. Tassi I, Presti R, Kim S, Yokoyama W M, Gilfillan S, Colonna M. PLCg2 is a critical signalling mediator for murine NK cell activating receptors. J Immunol. 2005; In press.
108. Gilfillan S, Ho E L, Cella M, Yokoyama W M, Colonna M. NKG2D recruits two distinct adapters to trigger NK cell activation and costimulation. Nat Immunol. 2002; 3:1150-1155.
109. Downward J. PI 3-kinase, Akt and cell survival. Semin Cell Dev Biol. 2004; 15:177-182.
110. Radsak M P, Salih H R, Rammensee H G, Schild H. Triggering receptor expressed on myeloid cells-1 in neutrophil inflammatory responses: differential regulation of activation and survival. J Immunol. 2004; 172:4956-4963.
111. Cella M, Buonsanti C, Strader C, Kondo T, Salmaggi A, Colonna M. Impaired differentiation of osteoclasts in TREM-2-deficient individuals. J Exp Med. 2003; 198:645-651.
112. Dietrich J, Cella M, Colonna M. Ig-like transcript 2 (ILT2)/leukocyte Ig-like receptor 1 (LIR1) inhibits TCR signalling and actin cytoskeleton reorganization. J Immunol. 2001; 166:2514-2521.
113. Saccani S, Pantano S, Natoli G. p38-Dependent marking of inflammatory genes for increased NF-kappa B recruitment. Nat Immunol. 2002; 3:69-75.
114. Piccio L, Vermi W, Boles K S, et al. Adhesion of human T cells to antigen-presenting cells through SIRP{beta}2-CD47 interaction costimulates T cell proliferation. Blood. 2004.
115. lizuka K, Naidenko O V, Plougastel B F, Fremont D H, Yokoyama W M. Genetically linked C-type lectin-related ligands for the NKRP1 family of natural killer cell receptors. Nat Immunol. 2003; 4:801-807.
116. Nakajima H, Cella M, Langen H, Friedlein A, Colonna M. Activating interactions in human NK cell recognition: the role of 2B4-CD48. Eur J Immunol. 1999; 29:1676-1683.
117. Feizi T. Carbohydrate-mediated recognition systems in innate immunity. Immunol Rev. 2000; 173:79-88.
118. Feizi T, Stoll M S, Yuen C T, Chai W, Lawson A M. Neoglycolipids: probes of oligosaccharide structure, antigenicity, and function. Methods Enzymol. 1994; 230:484-519.
119. Yamashita K, Ohkura T, Tachibana Y, Takasaki S, Kobata A. Comparative study of the oligosaccharides released from baby hamster kidney cells and their polyoma transformant by hydrazinolysis. J Biol Chem. 1984; 259:10834-10840.
120. Patel T P, Parekh R B. Release of oligosaccharides from glycoproteins by hydrazinolysis. Methods Enzymol. 1994; 230:57-66.
121. Townsend R R, Hardy M, Olechno J D, Carter S R. Chromatography of carbohydrates. Nature. 1988; 335:379-380.
122. Bredemeyer A J, Lewis R M, Malone J P, et al. A proteomic approach for the discovery of protease substrates. Proc Natl Acad Sci USA. 2004; 101:11785-11790.
123. Y. Murakami, Innate Immunity: Signaling Mechanisms, February 2008, Keystone, Colo.
124. Dower K, et al. Innate Immune Responses to TREM-1 Activation: Overlap, Divergence, and Positive and Negative Cross-Talk with Bacterial Lipopolysaccharide J. Immunol. 2008 180:3520-3534.
125. Haselmayer et al. TREM-1 ligand expression on platelets enhances neutrophil activation Blood 2007 110:1029-1035.
126. Michelson and Newburger Platelets and leukocytes: aggregate knowledge Blood 2007 110:794-795

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All references referred to in this application, including patents and patent applications, are incorporated herein by reference to the fullest extent possible as if each individual publication or patent application were specifically and individually indicated to be incorporated by reference.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1-47. (canceled)

48. A non human animal, wherein the animal is genetically modified for reduced expression of TREM-1 or of a TREM-1 ligand, relative to the wild type animal.

49. A non-human animal according to claim 48 that is a knock-out for a TREM-1 ligand or TREM-1 receptor.

50. A non-human animal, according to claim 48, wherein the animal is genetically engineered for reduced expression of TREM-3.

51. A non-human animal, according to claim 50 which is a TREM-1/TREM-3 double knock out rodent.

52.-65. (canceled)

66. An antibody that binds to a TREM-1 ligand so as to prevent the ligand binding to the TREM-1 receptor or to reduce the efficiency of such binding.

67. An antibody according to claim 66, that binds to the TREM-1 ligand, but does not bind to any other cell surface protein expressed on septic neutrophils or monocytes.

68. An antibody according to claim 66, that is specific for a TREM-1 ligand.

69. An antibody according to claim 68, that is specific for a part of the TREM-1 ligand that binds to a TREM-1 receptor.

70. An antibody according to claim 68, that binds preferentially to a mutant form of a TREM-1 ligand relative to a wild type TREM-1 ligand.

71. An antibody according to claim 68, that is specific for a mutant form of a TREM-1 ligand.

72. An antibody according to claim 68, that is specific for cells which present on their surface a TREM-1 ligand or a derivative thereof.

73. An antibody according to claim 66, wherein the TREM-1 ligand is CD177.

74.-87. (canceled)

88. A method for obtaining anti-TREM-1 ligand antibodies which comprises providing a TREM-1 ligand or a derivative thereof and using it to generate antibodies in a non-human host, or which comprises providing cells which present on their surface a TREM-1 ligand or a derivative thereof and using them to generate antibodies in a non-human host.

89. (canceled)

90. A method according to claim 88, further comprising the step of purifying the antibodies.

91. A method for obtaining a hybridoma producing anti-TREM-1 ligand antibodies comprising
a) providing a TREM-1 ligand or a derivative thereof;
b) using the ligand or derivative to generate a B cell that produces anti-TREM-1 ligand antibodies in a non-human host,
c) fusing the B cell with a tumour cell to produce the hybridoma.

92. A method according to claim 88, wherein the TREM-1 ligand or derivative is in substantially pure form.

93. A method according to claim 88, wherein the TREM-1 ligand is CD177.

94. A hybridoma that produces anti-TREM-1 ligand antibodies, said antibodies being antibodies that bind to a TREM-1 ligand so as to prevent the ligand binding to the TREM-1 receptor or to reduce the efficiency of such binding.

95.-97. (canceled)

98. A non-human animal according to claim 88 wherein the TREM-1 ligand is CD177.

99. A method according to claim 91 wherein the TREM-1 ligand is CD177.
